(19) United States
(12) Patent Application Publication
(10) Pub. No.: US 2019/0195883 A1
(43) Pub. Date: Jun. 27, 2019

(54) DIAGNOSTIC SEQUENCING VIA TABLETS

(71) Applicants: Warren Che Wor CHAN, Toronto (CA); Buddhisha Nayantara UDUGAMA, Toronto (CA); Pranav Karthike KADHIRESAN, Mississauga (CA)

(72) Inventors: Warren Che Wor CHAN, Toronto (CA); Buddhisha Nayantara UDUGAMA, Toronto (CA); Pranav Karthike KADHIRESAN, Mississauga (CA)

(21) Appl. No.: 16/181,679

(22) Filed: Nov. 6, 2018

Related U.S. Application Data
(60) Provisional application No. 62/582,072, filed on Nov. 6, 2017.

(51) Int. Cl. G01N 33/58; G01N 33/543

(52) U.S. Cl. CPC G01N 33/588; B82Y 15/00; G01N 33/54393

(57) ABSTRACT

A solid diagnostic product comprising reagents necessary for a protein-based test or genetic test and method of using said solid diagnostic product in detecting targets of interest in a fluid sample.

### CROSS-REFERENCE TO RELATED APPLICATIONS

This application claims the benefit of provisional application Ser. No. 62/582,072, filed Nov. 6, 2017, the full content of which is incorporated herein by reference.

### REFERENCE TO SEQUENCE LISTING SUBMITTED VIA EFS-WEB

This application includes an electronically submitted sequence listing in .txt format. The .txt file contains a sequence listing entitled “0226341.0002_ST25.txt” created on Feb. 27, 2019 and is 3,308 bytes in size. The sequence listing contained in this .txt file is part of the specification and is hereby incorporated by reference herein in its entirety.

### FIELD OF THE INVENTION

The present invention relates to the field of rapid multi-step diagnostic methods.

### BACKGROUND OF INVENTION

Settings with minimal laboratory infrastructure pose the greatest challenges to assay developers when designing point-of-care (POC) diagnostics for the developing world. First of all, these sites may be in perpetually hot locations that lack access to cold storage and transport, compromising the stability of diagnostic reagents. Most bioreagents used in POC assays, such as antibodies and enzymes, are susceptible to denaturation at elevated temperatures. In order to preserve the accuracy and the robustness of POC assays, these bioreagents need to be shipped in dry ice and stored in refrigerators to maintain thermal stability. Secondly, these sites may also have fewer health-care staff with the appropriate training to conduct complicated diagnostic assays, compromising test result accuracy. To avoid misdiagnosing disease conditions, assay developers need to work towards building simple diagnostic tests that require minimal operator-based intervention.

Quantum dot (QD) barcode assay is a platform that has temperature-sensitive reagents, while requiring multiple steps for completion of the assay. In a typical quantum dot (QD) barcode assay, target analytes are detected through a sandwich assay where microbeads conjugated with target-specific biomolecules hybridize with the target analyte and a fluorescent detection probe. QD barcodes, polymer microspheres infused with varying ratios of semiconductor quantum dots, offer a versatile platform for multiplexed biosensing applications.¹ The identity and the presence of the target analyte are then determined through the barcode and detection probe signal, respectively.² Although this platform offers numerous advantages for use at point-of-care, its temperature-sensitive beads and requirement of technical expertise can hinder its adoption in resource-limited settings. The bead structure is maintained by non-covalent hydrophobic interactions that can be easily disrupted under high temperatures. Previous studies have shown that under high heat conditions (>37° C.) barcodes degrade easily; resulting in a dramatic decrease in barcode fluorescence intensity.¹ Variability in the spectral profile of the barcodes can lead to incorrect identification of pathogens and misdiagnosis of disease conditions.³ In addition to its temperature-sensitive nature, the QD barcode assay has multiple steps required to detect clinical specimens. The QD barcode assay employs an isothermal amplification method, known as recombinase polymerase amplification (RPA), to detect clinical specimens with viral loads as low as 10³ copies/mL.⁴ This technique uses recombinase proteins that form a nucleoprotein complex with primers, to facilitate strand exchange at homologous sequences of the template DNA. As single-stranded binding proteins (SSB) stabilize this complex, a DNA polymerase extends the template of interest to provide exponential amplification. This amplified product is then used in the QD barcode assay to screen for specific diseases.⁴ This multi-step process can be time-consuming and require technical expertise to obtain accurate test results. Thus what is needed is a platform that can stabilize reagents for multi-step QD barcode assays while simplifying the multiple step diagnostic assay to a one-step assay.

### SUMMARY OF INVENTION

In one embodiment, the present invention provides for a solid diagnostic product comprising reagents necessary for the detection of a target of interest.

In one embodiment of the solid diagnostic product of the present invention, the reagents are of quantum dot barcodes conjugated to a capture probe having affinity for the target of interest.

In another embodiment of the solid diagnostic product of the present invention, the reagents are lyophilized in a sugar having high Tg and mixed with spray-dried mannitol and at least one acceptable carrier or excipient.

In another embodiment of the solid diagnostic product of the present invention, the solid diagnostic product is color-coded to mediate a diagnostic sequence.

In another embodiment of the solid diagnostic product of the present invention, the target of interest is a nucleic acid of interest, and wherein the solid diagnostic product includes: (a) a layer or tablet having reagents for amplifying a nucleic acid sequence of interest; (b) a layer or tablet having QD barcodes conjugated to a capture probe having affinity for the nucleic acid sequence of interest; (c) a layer or tablet having a reporter probe for the nucleic acid sequence of interest, and optionally (d) a layer or tablet having a reporter probe for controls. In one aspect of this embodiment, the (a) layer or tablet comprises reagents for a recombinase polymerase amplification (RPA). In another aspect of this embodiment, the reagents in the (a) layer or tablet are for RPA are: RPA recombinase, polymerase, single-stranded binding proteins (SSBs), and other co-factors and primers for the nucleic acid sequence of interest.

In one aspect of the present invention the primers reagents in the (a) layer or tablet include a 3′ C3 spacer between a hybridization and a primer sequence.

In another aspect of the present invention, the layers or tablets are color-coded to mediate a diagnostic sequence.

In another embodiment of the solid diagnostic product of the present invention, the solid diagnostic product includes the reagents lyophilized with trehalose and Iron-EDTA, mixed with spray-dried mannitol and sodium stearyl fumarate.

In another embodiment of the solid diagnostic product of the present invention, the solid diagnostic product includes: (a) a layer or tablet having a detectable primary ligand to the target of interest; and (b) a layer or tablet having a detectable secondary ligand to a complex formed by the primary ligand and the target of interest. In one aspect, the detectable primary ligand is a primary antibody-conjugated quantum dot (QD) barcode.

In another aspect the detectable secondary ligand is an AlexaFluor-647 dye conjugated secondary antibody.

In another embodiment of the solid diagnostic product of the present invention, the solid diagnostic product is a multi-layered tablet comprising: (a) a top, fast-release layer having a detectable primary ligands to the target of interest, (b) a core layer having secondary detection ligands to the complex formed between the primary ligand and the target of interest, and (c) a barrier layer between the top and the core layers configured to delay the core layer from releasing the secondary detection ligands.

In another embodiment of the solid diagnostic product of the present invention, the solid diagnostic product is a multi-layered tablet comprising: (a) a top, fast release layer having reagents for a recombinase polymerase amplification (RPA); (b) a core layer having QD barcodes and reporter probes; and (c) a barrier layer between the top and the core layers configured to delay the core layer from releasing the QD barcode components.

In one embodiment, the present invention provides for a kit for diagnosis of a condition produced by a pathogen, the kit comprising: a first solid diagnostic product comprising reagents for amplifying a DNA sequence characteristic of the pathogen, a second solid diagnostic product comprising quantum dot barcodes conjugated to a capture probe having affinity for the same DNA sequence of the pathogen, and a third solid diagnostic product comprising a labelled reporter probe having affinity for the same DNA sequence of the pathogen.

In one embodiment of the kit of the present invention the kit further comprises another solid diagnostic product comprising a reporter probe for controls.

In another embodiment of the kit of present invention, the reagents for amplifying the DNA sequence are reagents for a recombinase polymerase amplification.

In another embodiment of the kit of the present invention, the solid diagnostic product is provided in the form of a multi-layered tablet wherein each layer represents each of the solid diagnostic products.

In another embodiment of the kit of the first, second and third solid diagnostic products are color-coded to mediate the diagnosis of the condition.

In another embodiment, the present invention is a method of detecting a biological target of interest in a biological sample, the method comprising contacting the biological sample with the solid diagnostic product according to any of the embodiments of the present invention and analyzing the biological sample for a presence of the target in the biological sample.

In another embodiment, the present invention is a method of detecting a nucleic acid sequence of interest, the method comprising: (a) obtaining or providing a subject's biological sample (for example in fluid form), (b) purifying the nucleic acid in the sample thereby obtaining a solution with the purified nucleic acid, (c) adding a tablet according to an embodiment of the present invention containing nucleic amplification agents to the solution with the purified nucleic acid to amplify the nucleic sequence of interest, (d) adding to the solution (c) a tablet containing barcodes or nanoparticles having a conjugated probe with affinity to the nucleic sequence of interest, (e) adding a tablet containing a secondary probe having affinity to the complex formed between the conjugated probe and the nucleic acid of interest to the solution (d), and (f) analyze and detect the nucleic acid sequence of interest.

In another embodiment, the present invention is a method of detecting a target of interest in a biological sample, the method comprising: (a) obtaining or providing a subject's biological sample (for example in fluid form), (b) adding a multi-layered tablet according to an embodiment of the present invention and (c) analyzing and detecting the nucleic acid sequence of interest in the sample.

### BRIEF DESCRIPTION OF THE DRAWINGS

Embodiments will be described, by the way example only, with the reference to the drawings, in which:

FIGS. 1A to 1F. Screening chemical compounds for the development of reagent tablets. FIGS. 1A to 1C: Chemical structures of sugars maltose (FIG. 1A), sucrose (FIG. 1B) and trehalose (FIG. 1C) used to prevent stress-induced biomolecular degradation during lyophilization. FIG. 1D: i) DSC curves of heat flux vs. temperature for trehalose, maltose and sucrose. ii) Inset with T_{g} of trehalose. iii) Inset with T_{g} of maltose. iv) Inset with T_{g} of sucrose. FIG. 1E: T_{g} of trehalose after moisture uptake. FIG. 1F: Weight changes of tablets stored at 80% RH for 1 week.

FIGS. 2A to 2E. Development of compressed tablets FIG. 2A: Schematic representation of the development of diagnostic tablets FIG. 2B Photograph of compressed tablets of various sizes and colors (Scale bar: 1 cm). Compressed tablets are inexpensive to develop and the sizes and colors of the tablets can be customized for different types of assays. FIGS. 2C to 2E: Reagents are protected in tablets by using the stabilization properties of trehalose and the barrier environment created by the tablets. Trehalose is postulated to stabilize reagents by i) reducing their mobility in a glassy matrix (FIG. 2C), or ii) by hydrogen bonding with the reagents and acting as a substitute for water (FIG. 2D), or lastly, iii) by sequestering water and thereby reducing their interaction with water (FIG. 2E).

FIGS. 3A to 3F. Characterization of reagent tablets using quantum dot barcodes as a model diagnostic system. FIGS. 3A to 3C. Fluorescence microscopy images of barcodes before and after encapsulation in compressed tablets. Scale bar: 50 μm. The positions of the microbeads were determined using a 405/20 nm BP and 420 LP excitation and emission filters. FIG. 3A: fluorescence images before and after encapsulating 3.9 μm 435 nm QD microbeads in compressed tablets. Images were obtained using 360/40 BP excitation and 447/60 nm BP (blue filter set) emission filters. FIG. 3B: Composite images for two types of microbeads (435 nm and 586 nm) before and after encapsulating in compressed tablets. Microbeads were then imaged with both the blue filter set, as well as 480/40 BP excitation and 650/50 nm BP emission (red filter set). FIG. 3C: Composite images of three types of microbeads (435 nm, 586 nm and 525 and 575 nm) before and after encapsulating in compressed tablets. Microbeads were imaged with the blue and red filter sets as well as with 480/40 nm BP excitation and 580/10 nm BP emission filter sets. FIGS. 3D to 3E: Size distribution of respective microbeads illustrated in FIGS. 3A to FIG. 3C before and after encapsulation in compressed tablets.

FIGS. 4A to 4B. Characterization of thermal stability (4° C. and cycling conditions) for barcodes in solution (control) and in tablets via a single-plex sandwich assay. FIG. 4A. Time-based sensitivity curves for i) barcodes in solution at 4° C. and ii) cycling temperatures (4° C., 25 ° C., 37° C. every 2 days), and barcodes in tablets at iii) 4° C. and at iv) cycling temperatures for 12 weeks at 2-week time-points. FIG. 4B. Fluorescence microscopy images (excitation: 480/40 nm BP and emission: 580/10 nm BP) of microbeads in tablets and in solution (control) for 12 weeks at 2-week time points.

FIG. 5. Size distribution of QD barcodes in compressed tablets and in solution (control) at 2-week time points at 4° C., 25° C., 37° C. and cycling temperatures (cyclic storage at 4° C., 25° C., 37° C. every 2 days). Size distribution was analyzed using Vi-Cell Counter from Beckman Coulter. Statistical significance was determined by conducting a Mann-Whitney test at each time-point, comparing barcodes in solution and in tablets using IBM SPSS software. *=p<0.05.

FIG. 6. Characterization of thermal stability for reporter probes (oligonucleotide with AlexaFluor647) in solution (control) and in tablets at 25° C. and 37° C. via a single-plex sandwich assay. The thermal stability of reporter probes in solution and in tablet form were evaluated over time at different temperatures. At each time point, tablets were dissolved in 500 μL of TE buffer followed by subsequent filtration of excipients via a 0.22 μm filter and concentration of the filtrate (reporter probes) to 45 μL. Reporter probes in solution-form as well as in tablet-form were used in a sandwich assay with 0, 10, 25, 50, 100 fmol of HIV target DNA to create a sensitivity curve. The corresponding reporter probe signals were analyzed using flow cytometry. The median intensities were normalized to the highest signal of the respective 4° C. reporter probes in solution or tablets. Time-based (every 2 weeks) sensitivity curves of reporter probes stored in i) solution at 25° C. and ii) at 37° C. and in tablets iii) at 25° C. and iv) 37° C.

FIGS. 7A to 7B. Characterization of thermal stability for barcodes in solution (control) and in tablets via a single-plex sandwich assay. FIG. 7A. Fluorescence microscopy images (excitation: 480/40 nm BP and emission: 580/10 nm BP) of barcodes in tablets and in solution (control) for 12 weeks at 4-week time points. Over time, QD microbeads aggregate as increased temperature leads to the disruption of non-covalent forces that hold the polystyrene co-polymer of the microbeads. FIG. 7B. Time-based sensitivity curves for i) barcodes in solution at 25° C. and at ii) 37° C., and barcodes in tablets at iii) 25° C. and at iv) 37° C. for 12 weeks at 2-week time-points.

FIGS. 8A to 8D. Evaluating the mechanism of barcode stabilization in tablets. Barcodes were stored in i) 0.05% Tween 20 (FIG. 8A), ii) 15% trehalose (FIG. 8B), iii) lyophilized in 15% trehalose (FIG. 8C) and in iv) tablets (FIG. 8D) for 2 weeks at 4° C. and 37° C. The graph depicted illustrate the average reporter probe intensities of the barcodes at 37° C. A single-plex sandwich assay was conducted with each type of sample at 0, 3, 7, 10 and 14 days. 2 μL of barcodes of each type was mixed with 4 μL of water, 10 μL of hybridization buffer (10× SSC, 0.1% SDS) and 2 μL of 10 μM reporter probe. The average intensities for each sample at 37° C. were normalized to the respective average reporter probe intensities of the barcodes stored at 4° C. A 4-factor repeated measures ANOVA was conducted to compare the stability of the 4 types of barcodes (i.e. barcodes in 0.05% Tween 20, 15% trehalose, lyophilized in trehalose and in tablets) at 37° C. A post-hoc Bonferroni comparison was then conducted to determine the differences. Table 6 below illustrates the p-values obtained from this analysis. In summary, the main mechanism of stabilization of the barcodes is through the removal of water. Other mechanisms such as the densification of powder blends through tableting may also play a role in stabilization, however future work is required to probe these pathways.

FIGS. 9A to 9B. Characterizing reagent tablets for a protein and a genetic test. FIG. 9A. Time-based standard curves conducted for an immunoassay with avidin-horse radish peroxidase (avidin-HRP) compressed tablets stored at 25° C. for 4 weeks. At each time-point, avidin-HRP tablets were used to detect CXCLS chemokine in a sandwich immunoassay format. The optical density was measured using a plate reader. Statistical significance was determined with regression analysis using GraphPad Prism software. FIG. 9B. Time-based amplification of synthetic DNA using recombinase polymerase amplification tablets stored at 25° C. for 4 weeks. At each time point, RPA tablets were used to amplify water (negative control) and 10⁷ copies of synthetic DNA. A sandwich assay was then conducted with barcodes coated with capture probes complementary to the amplicons and a detection probe. The results were analyzed using flow cytometry. Statistical significance was calculated using a Mann-Whitney test using SPSS software.

FIGS. 10A to 10C. Screening for healthy and HBV+ patient samples using reagent tablets in a multi-step assay. FIG. 10A. Schematic representation of the workflow for validation. FIG. 10B. Results of screening healthy and HBV+ clinical samples with a bench-top flow cytometer. Compressed tablets encapsulated with diagnostic reagents were able to differentiate between healthy and HBV+ patient samples (p<0.0001) at bench-top. FIG. 10C. Results of screening healthy and HBV+ clinical samples with smartphone imaging using a wireless diagnostic device. Compressed tablets encapsulated with diagnostic reagents were able to differentiate between healthy and HBV+ patient samples (p<0.0001) at point-of-care. Statistical significance was determined by conducting a Mann-Whitney test between pooled healthy and HBV+ signal intensities in IBM SPSS software. LOD=limit of detection, **=p<0.01, ****<0.0001.

FIGS. 11A to 11C. Development of a multi-layered tablet for the RPA-QD barcode system. FIG. 11A. Schematic showing the steps for developing a multi-layered tablet. FIG. 11B Multi-layered tablets at different stages of development. Scale: 3 mm. FIG. 11C side and cross-sectional view of a multi-layered tablet according to one embodiment of the present invention.

FIGS. 12A to 12C. Design of “tailed primer” sequences. FIG. 12A. Schematic depiction of “tailed primers”. Tailed primers comprise of a primer sequence meant to amplify target sequence of interest, a C3 spacer to cease polymerase extension and a hybridization sequence for downstream sandwich assay. FIG. 12B. Schematic depiction of expected amplicon after amplification with tailed primers. At either ends of the amplicon, single-stranded overhangs are created. FIG. 12C. Sandwich-assay complex created as a result of tailed primers. Either ends of the single-stranded DNA hybridizes with the DNA-conjugated QD barcode and the fluorescent detection probe.

FIG. 13. Recombinase polymerase amplification (RPA) with tailed primers. L. low molecular weight ladder (25-766 bp). Lane 1 and 2—RPA with tailed primers and water (negative control) incubated at 37° C. for 60 minutes. Lane 3, 4, and 5—RPA with tailed primers and 10⁶ copies of DNA incubated at 37° C. for 30 minutes. Lane 6, 7 and 8—RPA with tailed primers and 10⁶ copies of DNA incubated at 37° C. for 60 minutes. Lane 9 and 10—RPA with tailed primers and water incubated at 42° C. for 60 minutes. Lane 11, 12, and 13—RPA with tailed primers and 10⁶ copies of DNA incubated at 42° C. for 30 minutes. Lane 14, 15 and 16—RPA with tailed primers and 106 copies of DNA incubated at 42° C. for 60 minutes.

FIGS. 14A to FIG. 14C. Primer designs used for validating the production of single-stranded o verhangs. FIG. 14A. Control primer set 1 (i.e. Cl FP/C1RP). Control primer set 1 only contains the primer sequence (therefore does not contain the C3 spacer of the hybridization sequence. The amplicons produced by RPA do not have single-stranded overhangs, therefore should no hybridize with dye-labeled single-stranded oligonucleotides (Cy5 and Cy3 labeled DNA) complementary to the hybridization sequence. FIG. 14B. Control primer set 2 (i.e. C2FP/C2RP). Control primer set 2 only contains the hybridization and primer sequence (therefore does not contain the C3 spacer). The amplicons produced by RPA do not have single-stranded overhangs, therefore should no hybridize with dye-labeled single-stranded oligonucleotides (Cy5 and Cy3 labeled DNA) complementary to the hybridization sequence. FIG. 14C. Tailed primer set (i.e. FP/RP). Tailed primers have a hybridization sequence, a C3 spacer, and a primer sequence. The amplicons produced by RPA have single-stranded overhangs to which the Cy5 and Cy3 labeled complementary DNA can bind to, producing dual fluorescent signals.

FIGS. 15A to 15C. Validating the development of single-stranded overhangs from tailed-primer RPA. FIG. 15A. SYBR gold stain, FIG. 15B. Cy3 signal and FIG. 15C. Cy5 signal for 1. Low-molecular weight ladder, 2. Cy5 oligonucleotide, 3. Cy3 oligonucleotide, 4. Negative control (i.e. water in place of target DNA) for hybridization assay, 5. Positive control (single-stranded target DNA), 6. RPA amplicons with tailed primers (have primer and hybridization sequence and C3 spacer), 7. RPA amplicons with control primer set 1 (only primer sequence, no hybridization and C3 spacer), 8. RPA amplicons with control primer set 2 (have both hybridization and primer sequence, no C3 spacer), 9. RPA amplicons with forward tailed primer (complementary to Cy3 oligo) and reverse control primer set 1. 10. RPA amplicons with forward tailed primer and reverse control primer set 2. 11. RPA amplicons with reverse tailed primer (complementary to Cy5 oligo) and forward control primer set 1. 12. RPA amplicons with reverse tailed primer (complementary to Cy5 oligo) and forward control primer set 2.

FIG. 16. Tailed-primer RPA with excipients. Primers and other RPA components were mixed and incubated at 42° C. for 60 minutes. L. Ladder, Lane 1 and 2. Tailed-primer RPA with no excipients, lane 3 and 4. Tailed-primer RPA with mannitol and Lane 4 and 5. Tailed-primer RPA with lactose.

FIG. 17A is a schematic of a multi-layered tablet according to one embodiment of the present invention.

FIG. 17B is a cross-section of a multi-layered tablet for nucleic acid amplification in accordance to one embodiment of the present invention. The top layer is comprised of amplification reagents while the core of the tablet is comprised of a layer of QD barcodes and a layer of reporter probes.

### DETAILED DESCRIPTION OF INVENTION

### Definitions

Unless defined otherwise, all technical and scientific terms used herein have the same meaning as commonly understood by one of ordinary skill in the art to which this invention belongs. Also, unless indicated otherwise, except within the claims, the use of “or” includes “and” and vice versa. Singular forms included in the claims such as “a”, “an” and “the” include the plural reference unless expressly stated otherwise. All relevant references, including patents, patent applications, government publications, government regulations, and academic literature are hereinafter detailed and incorporated by reference in their entireties. In order to aid in the understanding and preparation of the within invention, the following illustrative, non-limiting, examples are provided.

The term “comprising” means any recited elements are necessarily included and other elements may optionally be included. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

The term “about” modifying any amount refers to the variation in that amount encountered in real world conditions of producing materials such as polymers or composite materials, e.g., in the lab, pilot plant, or production facility. For example, an amount of an ingredient employed in a mixture when modified by about includes the variation and degree of care typically employed in measuring in a plant or lab producing a material or polymer. For example, the amount of a component of a product when modified by about includes the variation between batches in a plant or lab and the variation inherent in the analytical method. Whether or not modified by about, the amounts include equivalents to those amounts. Any quantity stated herein and modified by “about” can also be employed in the present invention as the amount not modified by about.

In this specification and in the claims that follow, reference will be made to a number of terms that shall be defined to have the meanings below. All numerical designations, e.g., dimensions and weight, including ranges, are approximations that typically may be varied (+) or (−) by increments of 0.1, 1.0, or 10.0, as appropriate. All numerical designations may be understood as preceded by the term “about”.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The term “ligand” or “probe” as used herein refers to a capture molecule, organic or inorganic, or group of molecules that exhibits selective and/or specific binding to one or more organic or inorganic targets. Targets may include molecules such as nucleic acids such as DNA and RNA, proteins, fatty acids, carbohydrates and so forth. Targets may also include specific sites of a receptor, a probe, or whole microscopic organisms (unicellular or multicelluar) such as a pathogen. There can exist more than one ligand for a given target. The ligands may differ from one another in their binding affinities for the target. Examples of ligands include nucleotide-based ligands (aptamers, oligonucleotides, and so forth), amino acid-based ligands (antibodies, peptides, proteins, enzymes, receptors and so forth), polysaccharide-based ligands (for example hyaluronan), antigens, hormones, including peptide-hormones, lipid/phospholipid-hormones and monoamine hormones, and any other molecule capable of binding to an organic or inorganic target.

Multiplex may be understood as the ability to detect the presence of more than one target simultaneously. The multiplex detection system may include barcodes, metal, semiconductor, or organic based nanostructures or molecules, (e.g. organic dyes).

Barcodes may include any type of structure or system that allows a target to be distinguished. Barcodes that may be used with the present invention include magnetic, optical (i.e. quantum dots, organic dyes), electrical, DNA and lithographic barcodes.

As used herein, a “quantum dot” (QD) is a semiconducting photoluminescent material, as is known in the art (For example, see Alivasatos, Science 271:933-937 (1996)). Non-limiting examples of QDs include: CdS quantum dots, CdSe quantum dots, CdSe/CdS core/shell quantum dots, CdSe/ZnS core/shell quantum dots, CdTe quantum dots, PbS quantum dots, and/or PbSe quantum dots. As is known to those of skill in the art, CdSe/ZnS means that a ZnS shell is coated on a CdSe core surface (ie: “core-shell” quantum dots). The shell materials of core-shell QDs have a higher bandgap and passivate the core QDs surfaces, resulting in higher quantum yield and higher stability and wider applications than core QDs.

Quantum dot barcodes refers to microbeads containing different combinations of fluorescent semiconductor nanocrystals. Each microbead may include a unique optical signature that identifies the surface conjugated molecule. Approximately 10,000 to 40,000 different optical barcodes may be engineered using 5-6 different color quantum dots and six intensity levels. This enables significant multiplexing and these barcodes can detect targets in a flow cytometer or microfluidic channel as well as through other means.

### Overview

The present invention relates to a solid diagnostic product, such as a diagnostic tablet, comprising reagents necessary for a multiple steps diagnostic assay, and to methods of diagnosis using said solid diagnostic product. The solid diagnostic product may be a multi-layered tablet or individual tablets, each tablet having one or more sets of reagents necessary for the multiple steps assay.

The solid diagnostic product of the present invention, in one embodiment, comprises the reagents necessary for a quantum dot (QD) barcode-based diagnostic assay. The solid diagnostic product may be a single, multi-layered tablet that includes all of the necessary reagents for the multiple step diagnostic assay, or it may be different, individual tablets, each tablet having one or more sets of reagents necessary for the multiple steps assay.

The solid diagnostic product of the present invention may be used to detect a target of interest, such as nucleic acids, proteins, fatty acids, carbohydrates, or any other biochemical compound.

As such, in one embodiment, a solid diagnostic product of the present invention may include (a) a layer or tablet having a detectable primary ligand to the target of interest, and (b) a layer or tablet having a detectable secondary detection ligand that binds to a complex formed by the primary ligand and the target of interest.

In the case of nucleic acids, in one embodiment, a solid diagnostic product of the present invention may include (a) a layer or tablet having reagents for amplifying a nucleic acid sequence of interest; (b) a layer or tablet having QD barcodes conjugated to a capture probe having affinity for the nucleic acid sequence of interest; (c) a layer or tablet having a reporter probe for the nucleic acid sequence of interest, and, optionally, (d) a layer or tablet having a reporter probe for controls. In one embodiment, the components of layers (b), (c) and (d) may be included in only one layer.

The nucleic acid sequence of interest may be a sequence relevant to a condition being diagnosed or tested, or it may be to test the presence of said nucleic acid sequence of interest in a sample. The nucleic acid may, in one embodiment, be DNA.

A multi-layered tablet for the detection of targets such as proteins, carbohydrates, fatty acids, may include a top layer (fast-release) having primary ligands such as antibodies to a target of interest and a core layer having secondary detection ligands, such as secondary antibodies to the complex formed between the target of interest and the primary ligand. The top layer and core layer can be separated by a barrier layer which will delay the core layer from releasing its contents, allowing for an incubation period. In the presence of a solution of target antigens, the top layer will first release the primary ligands. The barrier layer will delay the release of the core reagents, allowing for binding to occur. After a period of time (for example 1-2 hours), the core will release the secondary detection ligands, which will bind the ligand-bound target of interest, forming a detectable sandwich complex.

In another embodiment, the present invention is a method of screening or detecting a subject's sample for the presence of target of interest. In one embodiment, the method comprises: contacting the target of interest with a solid diagnostic product of the present invention.

In another embodiment, the method of screening or detecting the presence of a target of interest includes: (a) extracting, purifying or isolating the target of interest from the subject's sample, and (b) contacting the extracted, purified or isolated target of interest with a solid diagnostic product of the present invention.

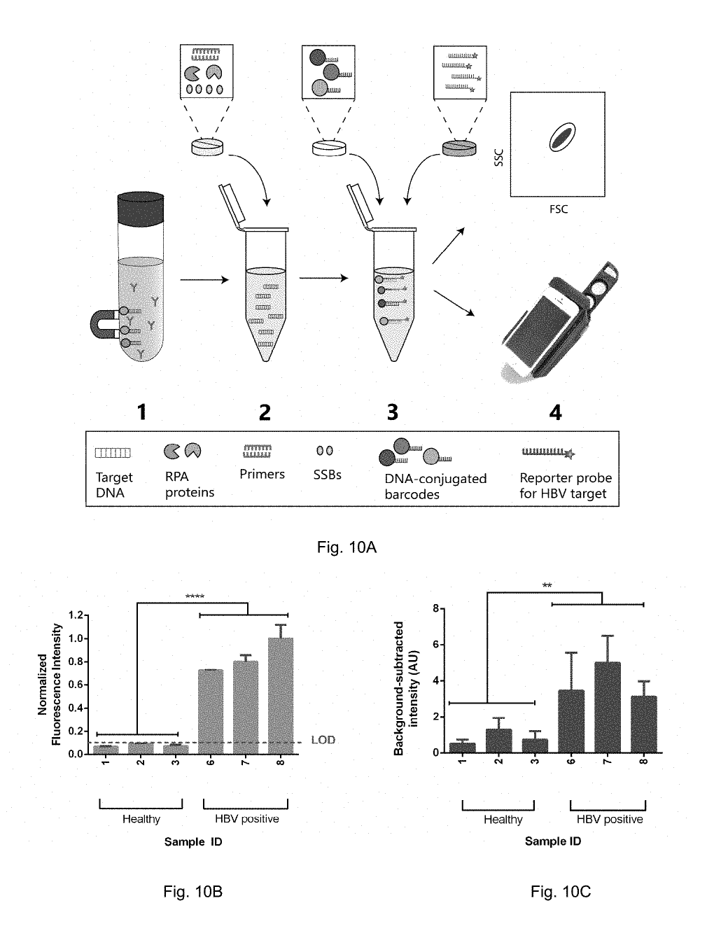

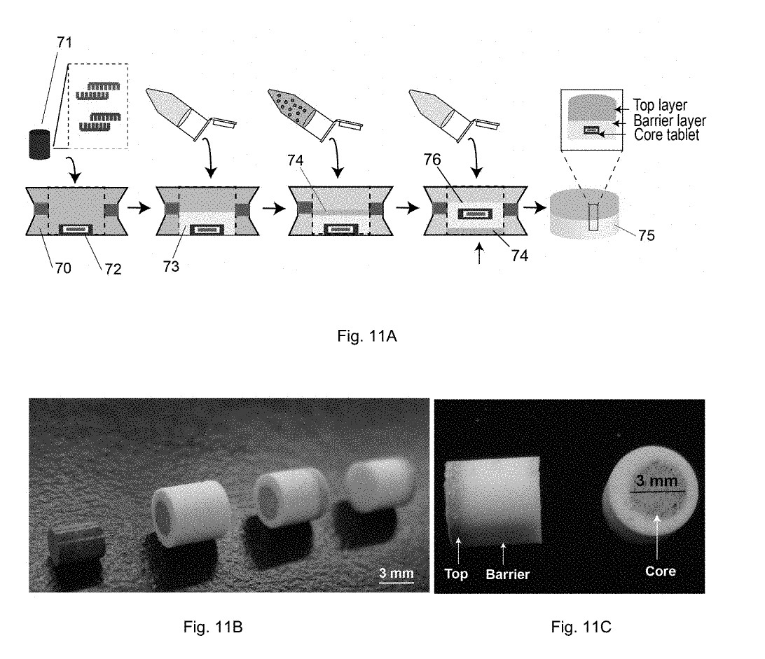

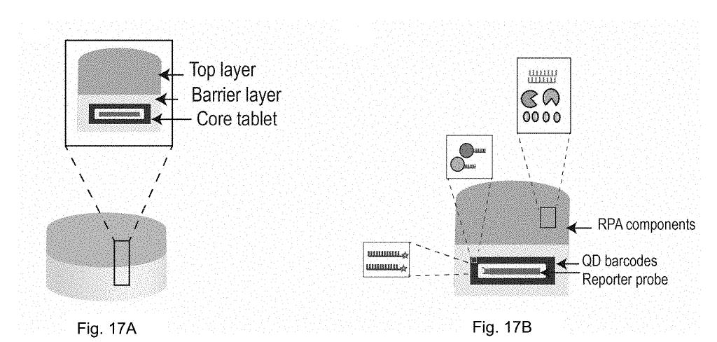

With reference to FIG. 10A, in another embodiment, the present invention is a method of screening a subject's sample for the presence of target nucleic acid comprising (a) extracting, purifying or isolating nucleic acids (NAs) from the subject's sample (item 1 of FIG. 10A), (b) contacting the extracted, purified or isolated NAs with a tablet containing reagents for amplification of the subject's nucleic acid (item 2 of FIG. 10A), (c) denaturing the amplified nucleic acid in the sample (for example by heating the sample at around 100° C.), and (d) adding a DNA-conjugated QD barcode tablet having affinity for the target nucleic acid and a tablet containing a reporter probe for the same target nucleic acid (item 3 of FIG. 10A). The final step is to analyze and detect the DNA sequence of interest in the solution containing the DNA-conjugated barcode (item 4 of FIG. 10A). The method of FIG. 10A can, in another embodiment be performed by a single, multi-layered tablet, such as the one embodied in FIG. 11A, and in FIGS. 17A,B. As explained below, when using the multi-layered tablet, the denaturing step may be avoided by using a modified RPA assay described below.

For detection of a DNA sequence of interest, a method according to the present invention using multiple single tablets may include:

(a) obtain or provide a subject's biological sample (preferably in fluid form), (b) extract, purify or isolate the DNA in the sample to obtain a solution with the extracted or purified or isolated DNA, (c) add a tablet containing amplification agents to the solution with the extracted, purified or isolated DNA to amplify the DNA sequence of interest, (d) incubate the solution with the amplified DNA sequence of interest, (e) add a tablet containing barcodes or nanoparticles having a conjugated probe with affinity to the incubated solution with the DNA sequence of interest, (f) incubate the solution with the barcodes or nanoparticles having the conjugated probe (g) add a tablet containing secondary probe with affinity for the same DNA sequence of interest to the incubated solution of (f), and (h) analyze and detect the DNA sequence of interest in the solution containing the secondary probe.

The incubation of steps (d) and (f) may be done at a temperature of 25-42° C., preferably 37-42° C., preferably at 37° C., for about 15 to 60 minutes (preferably 30 minutes).

Each tablet or each layer, as the case may be, may be colour-coded to identify each of the steps in the diagnostic procedure.

The tablet procedure would contain defined concentrations of each molecular or nanotechnology component.

The tablet or tablets may be designed to carry a single or multiple sensors (or barcodes) to enable the detection of either a single or multiple molecules with the correct ratio's. For example, the tablet(s) may contain 1 million or more of green-emitting barcodes and 1 million or more of red-emitting barcodes. The precision of carrying equal numbers of barcodes would influence the accuracy and reproducibility of the diagnostic process.

### RPA

In one embodiment, the present invention employs an isothermal amplification method, known as recombinase polymerase amplification (RPA), to detect clinical specimens with viral loads as low as 10³ copies/mL. This technique uses recombinase proteins that form a nucleoprotein complex with primers, to facilitate strand exchange at homologous sequences of the template DNA. As single-stranded binding proteins (SSB) stabilize this complex, a DNA polymerase extends the template of interest to provide exponential amplification. This amplified product is then used in the QD barcode assay to screen for specific diseases. As such, the layer or tablet for amplifying the DNA of interest, may include proteins necessary for RPA including recombinase, polymerase, single-stranded binding proteins (SSBs) and other co-factors.

### Development of Individual Tablets

Tablets can be created by first lyophilizing the diagnostic reagents. The lyophilized reagents can then be mixed with additives and this mixture can then be compressed into individual tablets.

The lyophilisation may be made in the presence of a sugar with a high glass transition temperature (Tg) such as trehalose, which was found to provide appropriate stabilization of the reagents. Other sugars that can be used include maltose and sucrose.

To compress the reagents into tablets, the lyophilized reagents can then be mixed with powders that can be compacted. Excipients that can be used for mixing with the lyophilized reagents include spray-dried mannitol, croscarmellose sodium and sodium stearyl fumarate. These excipients can be used alone or in combination. Other excipients may include D-sorbitol and microcrystalline cellulose.

After mixing the reagents and excipients, the mixture is compressed, for example with a rotary tablet press, using a compression force of about 800 N for barcodes mixtures and about 100 N for mixtures having biological materials. For example, 100 N can be used as compression forces for RPA or for mixtures containing avidin-horse radish peroxidase.

### Development of Multi-Layered Tablet

FIG. 11A illustrates steps for development of a multi-layered tablet in accordance to one embodiment of the present invention.

To a die mold 70, a pre-made core tablet 71 is placed at the center of the mold 70 thereby forming a core layer 72. The core layer 72 will contain the QD barcode components. The core layer 72 is then covered by a barrier layer 73. The barrier layer 73 provides time delay for RPA incubation. A fast-dissolving top layer 74 containing the RPA components is added, and a bottom barrier layer 76 is added below the core layer 72. The barrier layers 73, 76 may form a uniform barrier layer around the core layer 72. The layers are then compressed to form a multi-layered tablet 75. FIG. 11B-D are photographs of a multi-layered tablet and its cross-section (FIG. 11B), and multi-layered tablets at different stages of development (FIGS. 11C-D). A reporter probe can be mixed with the QD barcode components. Since, as shown in the Examples below, both the QD barcodes and the reporter probes are thermally stable in tablets, the two can be combined into one layer.

As shown in FIG. 11A, the RPA components are encapsulated in the top layer 74 while the QD barcode components are in the core layer 72. The top layer 74 with the RPA proteins will result in fast release (<30 seconds), followed by a time delay (for example 30-40 minutes) introduced by the barrier layer. The time delay will allow to provide the incubation time for RPA. After about 30 minutes, the core layer 72 will dissolve releasing the QD barcode components. Once released, the mixture will be left to complete the sandwich assay, for example for about 30 more minutes.

Amplicons, or amplified double-stranded target DNA, must be first denatured to single-stranded DNA, prior to their use in the QD barcode assay. To this end, the RPA assay has been modified such that the amplicons can be directly used with the QD barcode assay (without the need of denaturation step).

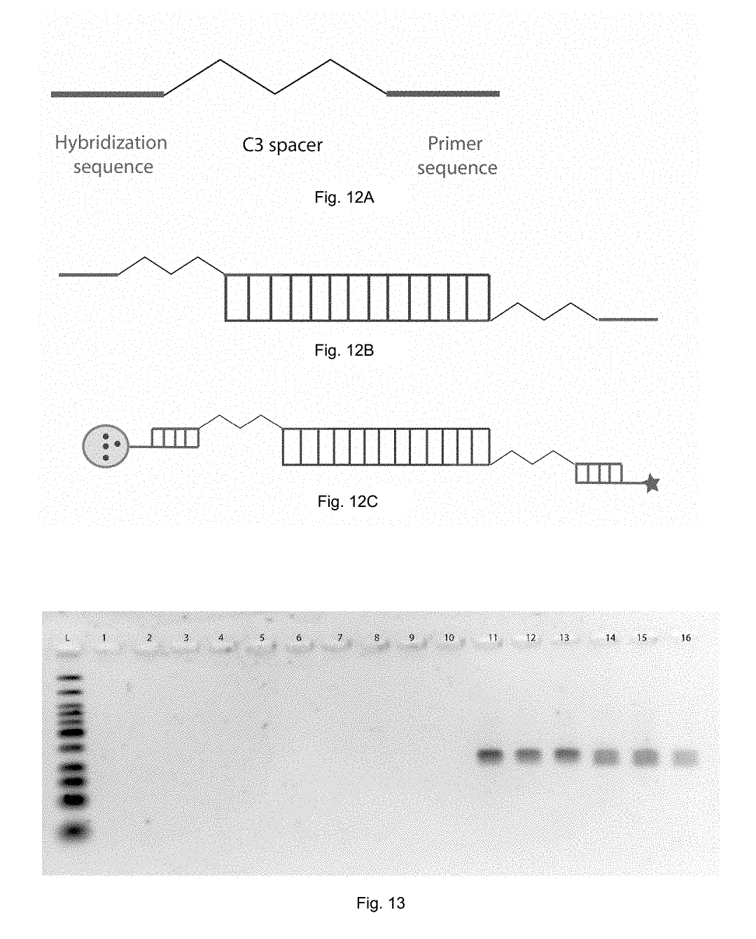

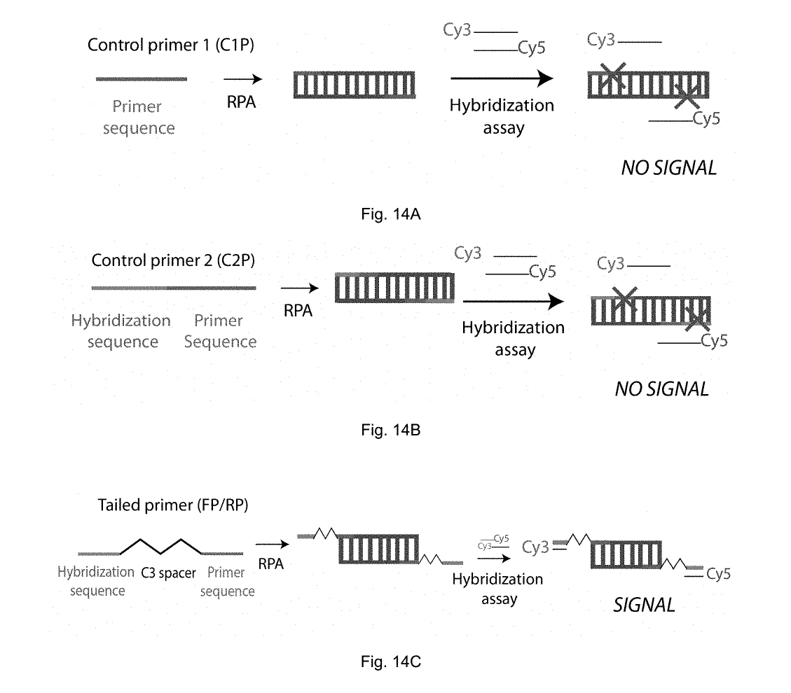

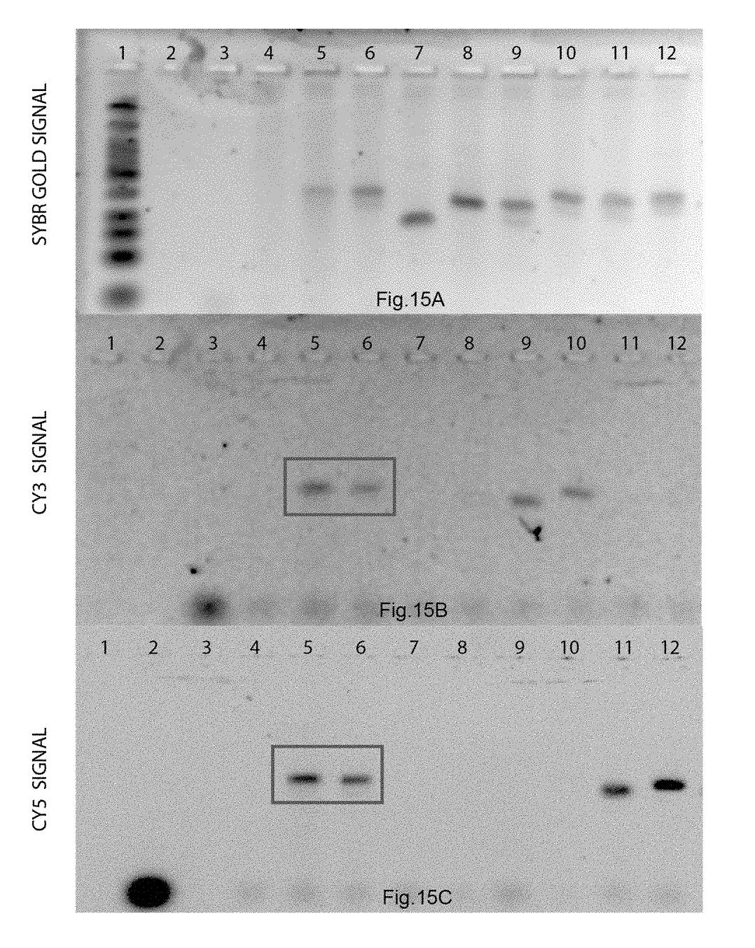

To ensure that the amplicons are directly compatible with the QD barcode assay, the primers were modified to include a 3′ C3 spacer sandwiched between a hybridization and a primer sequence (termed “tailed primers”) (FIG. 12A). When the polymerase encounters the spacer, elongation of DNA ceases, producing single-stranded overhangs on either end to be used directly for the QD barcode assay. The inventors were able to demonstrate that at 42° C., RPA was able to be performed using the tailed-primers (FIG. 13). In order to validate that the resulting product has single-stranded overhangs, a hybridization assay was conducted with Cy3 and Cy5-labeled oligonucleotides complementary to either ends of the amplicon. This assay demonstrated that the amplicons do in fact have single-stranded overhangs on either ends of the amplicon for downstream hybridization assays (FIGS. 14A to 14C and 15A to 15C).

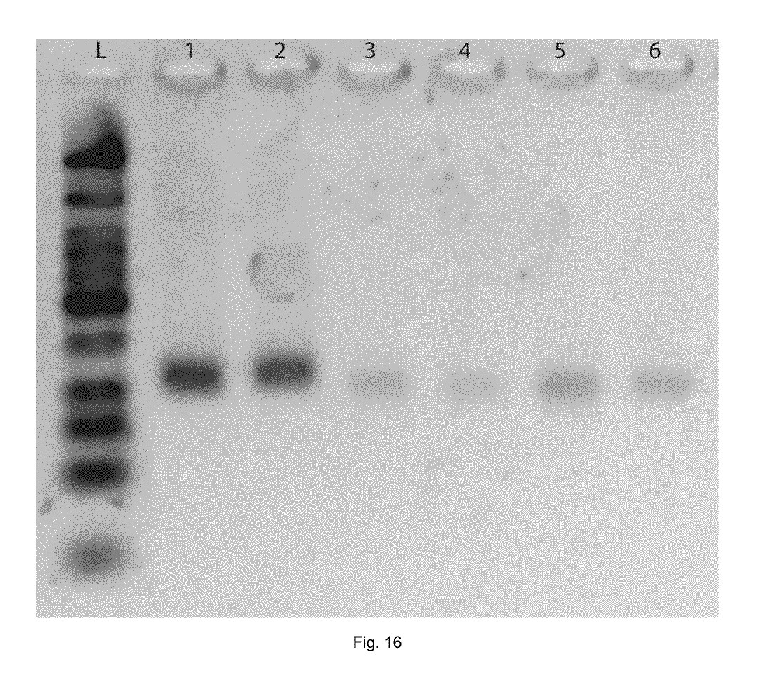

The tailed primers were mixed with excipients, mannitol and lactose, to evaluate primer-excipient compatibility. Among the two excipients, lactose provided a higher amount of product (qualitatively based on band intensity) than mannitol (FIG. 16).

A representative formulation for a multi-layered or single tablets combination would preferably have the following parameters:
Barcodes or nanoparticles are preferably less than 1% A by weight of the total multi-layered tablet or combination of single tablets.
Barcodes and/or nanoparticles are preferably polar. In order for the barcodes and/or nanoparticles to be better protected by the high Tg sugar such as trehalose during lyophilization, the reagents would preferably have the capability to form hydrogen bonds with the high Tg sugar.
Excipient (e.g., Mannitol) would preferably be >70%
Biological molecules, including DNA, proteins, antibodies and enzymes, may be less than 20% of the single multi-layered tablet or combination of single tablets.

Examples of tablets would be:

i. Nucleic Acid

Single Tablets (Not Multi-Layered):

Diagnostic reagents lyophilized with 15% trehalose in 0.05% Tween 20, 92.5% spray-dried mannitol, 6% croscarmellose sodium (optional) and 1% sodium stearyl fumarate.

Multi-Layered Tablet:

Fast release and core layers are comprised of diagnostic reagents lyophilized with 15% trehalose in 0.05% Tween 20, 78.5% spray-dried mannitol, 20% croscarmellose sodium (optional) and 1% sodium stearyl fumarate.

Barrier layer is comprised of 4-parts hydroxypropyl cellulose—SL grade to 1-part spray-dried mannitol.

ii. Non-Nucleic Acid Targets

Single Tablet (Not Multi-Layered)

In the case of targets other than nucleic acids, such as proteins, the single tablet can include antibodies or enzymes lyophilized with 15% trehalose in 0.05% Tween 20 and 7.5% of 0.133 M Iron-EDTA, mixed with 80% spray-dried mannitol and 2% sodium stearyl fumarate. For example, the single tablet (i.e. non-layered) may include avidin-horse radish peroxidase (avidin-HRP) lyophilized with 15% trehalose in 0.05% Tween 20 and 7.5% of 0.133 M Iron-EDTA, mixed with 80% spray-dried mannitol and 2% sodium stearyl fumarate.

Multi-Layered Tablet

A multi-layered tablet for the detection of targets other than nucleic acids, such as protein may include a top layer (fast-release) having detectable primary ligands to the target of interest, such as antibodies or antibody-conjugated to QD barcodes, and a core layer having secondary detection ligands to the complex formed between the primary ligand and the target of interest. The top layer and core layer will be separated by a barrier layer which will delay the core layer from releasing its contents, allowing for an incubation period. In the presence of a solution of target antigens, the top layer will first release the primary antibodies. The barrier layer will delay the release of the core reagents, allowing for binding to occur. After a period of time (1 to 2 hours), the core will release the secondary detection antibodies, which will bind the antibody-bound antigens, forming a sandwich complex.

In one embodiment, the multi-layered tablet may include a top layer (fast-release) having antibody-conjugated quantum dot (QD) barcodes and a core layer having AlexaFluor-647 dye conjugated secondary antibody. The top layer and core layer will be separated by a barrier layer which will delay the core layer from releasing its contents, allowing for an incubation period. In the presence of target antigens, the top layer will first release the antibody-conjugated QD barcodes, allowing the antigens to be bound to the antibodies on the barcodes. The barrier layer will delay the release of the core reagents, allowing for binding to occur. After a period of time (1 to 2 hours), the core will release the AlexaFluor-647 dye conjugated secondary antibody, which will bind the antigens, forming a sandwich complex. The QD barcode signal will provide the identity of the antigen while the AlexaFluor-647 dye will indicate the presence or absence of the antigen.

The invention will be further understood from the following examples, which are intended to be illustrative and not limiting of the scope of the invention.

### EXAMPLES

### 1. Materials, Trade Names and Manufacturers

Determination of glass transition temperature. 15% w/v D-trehalose dihydrate (University of Toronto MedStore) in 0.05% Tween 20 (BioShop Canada), 15% w/v D-maltose (University of Toronto MedStore) in 0.05% Tween 20 and 5% w/v D-sucrose (Sigma-Aldrich) in 0.05% Tween 20 were dipped in liquid nitrogen for ˜2 minutes and lyophilized (Labconco Free Dryer) overnight at ˜5 μHg. The glass transition temperature of lyophilized powder was then evaluated using a differential scanning calorimeter (Q2000 Differential Scanning calorimeter, NanoMed Fab Facility). The samples were first heated above each sugar's theoretical Tg⁵ at a heating rate of 10° C./min, then the samples were cooled to 25° C. at 5° C./min, then heated to 220° C. at a rate of 10° C./min⁶. The glass transition temperature was then determined using TA Universal Analysis software.

Moisture uptake study. Three replicates of 760 mg of D-sorbitol (University of Toronto MedStore), spray-dried mannitol (Mannogem EZ Spray Dried, SPI Pharma) and microcrystalline cellulose (Avicel PH-102 gifted by Dr. David Dubins) were placed in a controlled humidity chamber at 80% relative humidity. Six replicate tablets were made from the aforementioned powders by compressing the excipients at 800N using a rotary tablet press (GlobePharma Inc). The tablets were then stored at 80% RH and the mass of the tablets (AL54 Mettler Toledo) were determined every day for 1 week.

QD synthesis. 435 nm cadmium zinc sulfide/zinc sulfide QDs and 586 nm cadmium selenide/zinc sulfide QDs were synthesized as per established protocols^{7,8}.

Magnetic nanoparticle (FeO) synthesis. Iron (II) oxide (FeO) nanoparticles were synthesized via thermal decomposition of iron (III) acetylacetonate with oleic acid and oleylamine as per established protocols⁹.

QD Barcode synthesis. QD microbeads were synthesized via concentration-controlled flow focusing method as per our previous work³. Briefly, 400 mg of poly(styrene-co-maleic anhydride) (32% cumene terminated, Sigma-Aldrich) was mixed with 10 mL of chloroform and filtered with 0.22 pm PTFE filters (Nalgene). Several combinations of 435 nm (synthesized), 586 nm (synthesized), 525 nm (Crystalplex) and 575 nm (Crystalplex) QDs were mixed with 450 μL of 36.5 ng/mL of FeO magnetic nanoparticles and 3 mL of the aforementioned polymer solution (Table 1). The polymer and deionized water were then used as focused and focusing fluid at a rate of 0.9 mL/hr and 180 g/hr respectively to concentrate the solutions through a nozzle system (Ingeniatrics) using a syringe pump system (Harvard Apparatus) and a flow focusing controller (Hoskin Scientific).

Conjugation of single-stranded probes to QD barcodes. The single-stranded probes encompassed gene fragments for HIV, HBV, syphilis and gonorrhea DNA as previously described (Table 2). For clinical studies, the probes were designed to encompass HBV (Table 4). The probes were purchased from Bio Basic, Inc. The 5′ termini of the probes were amine modified (—NH2) and had a C6 spacer. The probes were conjugated to the surface of the microbeads via 1-ethyl-3-(3-(dimethylamino)propyl) carboiimide hydrochloride (EDC) chemistry. HIV, HBV, syphilis and gonorrhea capture probes were conjugated to B29, B33, B35 and B30 respectively. Briefly, 462 million beads were mixed with 31.42 mL of MES buffer (pH 5, 100 mM) and 14.78 mL of 0.30 g/ mL EDC (Chem-Implex). After incubation for 15 minutes, the supernatant was removed. 1.33 nmol of capture probe DNA (1.30 mL of 10 pmol/μL), 4.62 mL of 0.10 g/mL N-hydroxysulfosuccinimide (Chem-Implex) and 40.63 mL of bicarbonate buffer (pH 8, 100 mM) were added to the pellet and left for incubation overnight. After incubation, the conjugated beads were washed three times with 0.05% Tween 20.

Compressed tablet development. Thermal stability studies with QD barcode tablets. All four barcode types were combined in one tube and mixed with 33 mL of 15% trehalose in 0.05% Tween 20. The mixture was dipped in liquid nitrogen for ˜2 minutes and lyophilized overnight at ˜5 μHg. The barcodes were then mixed with 5 g of spray-dried mannitol (Mannogem EZ Spray Dried, SPI Pharma), 324 mg of croscarmellose sodium (Tablet Press Club) and 54 mg of sodium stearyl fumarate (Tablet Press Club). The mixture was ground with a mortar and pestle and filtered through a #40 mesh sieve (McMaster-Carr). 200 mg tablets were then developed by compacting the powder blends in a rotary tablet press (GlobePharma Inc) using a compression force of 800 N. Thermal stability studies with avidin-HRP tablets. For each tablet, 250 μL of avidin-HRP from Human CXCLS (ENA-78) kit (BioLegend) was mixed with 120 μL of 50% trehalose in 0.05% Tween 20 and 30 μL of 0.133 M Fe-EDTA. The mixture was frozen and lyophilized as aforementioned and mixed with 40 mg of spray-dried mannitol and 1 mg of sodium stearyl fumarate. ˜50 mg tablets were developed by compressing the mixture at 100 N using a rotary tablet press. Thermal stability studies with RPA tablets. For each RPA tablet, 12 μL of 10 pM forward and reverse primer (Bio Basic Inc.) (Table S7) were lyophilized with 280 μL of 15% trehalose in 0.05% Tween 20. The lyophilized mixture was then mixed with 4 RPA pellets (TwistDx), 41.2 mg of spray-dried mannitol, 3 mg of croscarmellose sodium and 1 mg of sodium stearyl fumarate. ˜70 mg tablets were developed by compressing at 100 N using a rotary tablet press. Clinical studies. Disease-specific RPA tablets were synthesized by first lyophilizing 912 pmol each of forward as well as reverse primers (Table S7) for HBV (Bio Basic Inc.) with 2.1 mL of 15% trehalose in 0.05% Tween 20. The primers were then mixed with 36 RPA pellets (TwistDx), 567 mg of spray-dried mannitol, 41 mg of croscarmellose sodium, and 7 mg of sodium stearyl fumarate. The mixture was compressed at 100 N to synthesize ˜50-60 mg tablets capable of running 4 amplification reactions. QD barcode tablets were synthesized by lyophilizing disease-specific barcodes conjugated with HBV ssDNA with 648 μL of 15% trehalose in 0.05% Tween 20. The lyophilized barcodes were then mixed with 266 mg of spray-dried mannitol, 17 mg of croscarmellose sodium and 3 mg of sodium stearyl fumarate. The mixture was compressed at 100 N to produce 4 ˜60 mg tablets. Likewise, reporter probe tablets, containing 2 nmol of HBV clinical reporter probe lyophilized in 1.4 mL of 15% trehalose in 0.05% Tween 20, were synthesized with the same proportion of powders.

Thermal stability studies: Single-plex sandwich assay. Barcodes in solution and in tablets were stored at 25° C. and 37° C. for 12 weeks (for 4° C. and cycling temperatures refer to supplementary information). Barcode tablets were resuspended in 0.05% Tween 20, washed three times using 0.05% Tween 20 using a magnetic rack (MagnaRack, Life Technologies) and barcode concentrations were normalized with Vi-Cell Cell Counter. Barcodes stored in solution and barcodes in tablets were then used in a sandwich assay using B29-HIV as a model system. The detection probe was purchased from IDT DNA Technologies and the target probes were purchased from BioBasics Inc (Table S2). The detection probe was labeled with Alexa Fluor 647 in its 3′ terminus. For each assay reaction, a premix containing 2 μL of detection probe (10 pmol/μL), 10 μL of hybridization buffer (10× SSC, 0.1% SDS heated to 60° C.), 2 μL of target DNA (either 1× TE buffer, 5 fmol/μL, 10 fmol/μL, 25 fmol/μL, or 50 fmol/μL of HIV target DNA) and 4 μL of water. 18 μL of the premix was then mixed with 2 μL (˜10,000 beads/μL) of the respective barcodes. The mixture was then incubated at 37° C. for 30 minutes and washed three times with 200 μL of the washing buffer (0.5× SSC, 0.1% SDS) using a magnetic rack. The final solution was resuspended in 200 μL of 1× PBS in 0.05% Tween 20 and analyzed using flow cytometry (BD FACSCalibur).

Thermal stability studies: Fluorescence microscopy of QD barcodes. All barcodes were normalized to 10,000 barcodes/μL and imaged using an Olympus fluorescence microscope with a 20× objective lens, 480/40 nm BP excitation filter and a 580/10 nm BP emission filter.

Thermal stability studies: ELISA with avidin-HRP tablets. Avidin-HRP tablets and 200 μL of avidin-HRP in solution were stored at room temperature (25° C.) for 4 weeks. At 2-week time points, a standard curve was created to evaluate stability. Avidin-HRP tablets were resuspended in 200 μL of water. Each tablet is capable of 2 reactions (100 μL each). Pre-coated anti-human CXCLS plates (BioLegend) were first washed four times with 1× wash buffer. Triplicate wells were coated with either 100 μL of Assay Buffer B (negative control), 100 μL of 31.25 pg/mL, 100 μL of 62.5 pg/mL and 100 μL of 125 pg/mL. The plate was incubated at room temperature for 2 hours while stirring, followed by a four wash steps. Next, 100 μL of human CXCLS detection antibody was added to each well and incubated at room temperature for 1 hour under stirring. Following incubation, the wells were washed four times with 1× wash buffer and 100 μL of avidin-HRP in solution or in tablets were added to the wells. The plate was then incubated for 30 minutes at room temperature under stirring, followed by five wash steps with 1-minute incubation between washes. 100 μL of Substrate F was added, followed by a 10-minute incubation. Lastly, 100 μL of the stop solution was added to each well. The optical density was measured at 450 nm and 570 nm (for reference) using Sunrise (Tecan) microplate reader.

Thermal stability studies: RPA tablets. To prepare solution-based RPA, each pellet was resuspended in 2.4 μL of forward and reverse primer (for a total of 4.8 μL) and 9.2 μL of water. RPA in tablets and in solution were stored at 25° C. for 4 weeks. At each time point, each RPA tablet was resuspended with 64 μL of water, 118 μL of rehydration buffer and 10 μL of 280 mM of magnesium acetate. Each tablet was capable of carrying out four amplification reactions. For each RPA reaction in solution, 2 μL of water, 29.5 μL of rehydration buffer and 2.5 μL of 280 mM magnesium acetate was added. 2 μL of 10⁷ synthetic DNA copies was included for amplification. After amplification, the amplicons were detected using the QD barcode sandwich assay. Briefly, as previously mentioned 2 μL of 10,000 beads/μL of QD barcodes coated with complementary probes (Table 4) was mixed with 2 μL of 10 μM reporter probe (Table 4), 10 μL of hybridization buffer (10× SSC, 0.1% SDS) and 1 μL of water. 5 μL of the amplicon was denatured at 100° C. and included in the reaction mixture. The samples were then incubated at 37° C. for 30 minutes and washed three times using wash buffer (0.5× SSC, 0.1% SDS). The samples were then detected using flow cytometry (BD FACSCalibur).

HBV clinical sample collection and DNA extraction from serum samples. HBV de-identified and healthy clinical samples were generously donated by Dr. Jordan Feld Lab, Toronto General Research Institute, Toronto, ON. The samples were obtained as per established protocols from the Toronto Western Hospital Liver Clinic, where the procedure was approved by the Research Ethics Board of University Health Network¹⁰. Patient samples were collected via venipuncture into a Vacutainer. The sample was kept upright for 30-60 minutes. Samples were centrifuged at 4° C. and stored at −80° C. Viral HBV and healthy DNA was extracted with Chemagic Viral DNA/RNA Kit (PerkinElmer).

Clinical recombinase polymerase amplification with tablets. A premix with 64 μL of water, 118 μL of rehydration buffer and 10 μL of 280 mM magnesium acetate was used to resuspend RPA tablets (capable of carrying out 4 reactions). 48 μL of the resuspended mixture was then used to amplify 2 μL of HBV or healthy patient samples. Triplicate tablets were used to amplify each of the clinical specimens. The RPA mixture was incubated at 37° C. for 1 hour and the amplicons were purified using spin columns (EZ-10 Spin Column DNA Gel Extraction Kit, Bio Basic Inc.). Successful amplification was verified using a 3% agarose gel.

Clinical validation: Single-plex sandwich assay. Benchtop. A barcode tablet, with three types of barcodes for the positive and negative control as well as HBV, was resuspended in 0.05% Tween 20 and concentrations were normalized with Vi-Cell Cell Counter. Two reporter probe tablets, one for the controls and one for HBV, were resuspended in 200 μL of 1× TE buffer. The amplified HBV DNA was denatured at 100° C. for 10 minutes. For 1 μL of each amplified DNA sample, 10 μL of heated hybridization buffer (10× SSC, 0.1% SDS), 3 μL of water, 1 μL from each of the reporter tablets (for the controls and the HBV samples), 1 μL of 100 fmol/μL syphilis synthetic DNA (Table S2), 1 μL of 1× TE buffer, and 2 μL of resuspended barcode tablet were added. The mixture was then incubated at 37° C. for 30 minutes and washed three times with 200 μL of the washing buffer (0.5× SSC, 0.1% SDS) using a magnetic rack. The final solution was resuspended in 200 μL of 1× PBS in 0.05% Tween 20 and analyzed using flow cytometry (BD FACSCalibur). Point-of-care (Smartphone imaging). A barcode tablet, with barcodes for HBV, was resuspended in 0.05% Tween 20 and concentrations were normalized with Vi-Cell Cell Counter. A reporter probe tablet for HBV was resuspended in 200 μL of 1× TE buffer. The amplified HBV DNA was denatured at 100° C. for 10 minutes. For 5 μL of each amplified DNA sample, 10 μL of heated hybridization buffer (10× SSC, 0.1% SDS), 1 μL of water, 2 μL of the reporter tablet and 2 μL of resuspended barcode tablet were added. The mixture was then incubated at 37° C. for 30 minutes and washed three times with 200 μL of the washing buffer (0.5× SSC, 0.1% SDS) using a magnetic rack. The final solution was resuspended in 10 μL of washing buffer and analyzed using smartphone imaging.

Smartphone imaging for point-of-care. Data collection and analysis was accomplished using smartphone imaging and MATLAB analysis of the images. Smartphone imaging included fluorescent microscopy of the barcodes using the device, and subsequent image collection using the ImageJ app on an IPhone 5s. The microscopy hardware was a combination of a PLA case 3-D printed using a Makerbot Replicator 2 that encased optical excitation, imaging and filtering elements. A 405 nm 50 mW laser diode module excited the barcodes. The Alexa Fluor reporter probe by a 655 nm 50 mW laser diode whose output was filtered using a 655/15 nm bandpass filter. Imaging was accomplished using a combination of three single element lenses acting as the objective (f=3.1 mm, NA=0.68), tube lens (f=11.0 mm) and eyepiece (f=4.03 mm) respectively. Emitted light was filtered using a 430 nm longpass filter and 692/40 nm bandpass filter to obtain barcode and reporter probe signal respectively. The barcodes were deposited on a glass MicroPEP slide and imaged using the ImageJ app, where ISO was set at 4000 and exposure at 20 seconds+/−2second.

Data analysis. Thermal stability studies. The flow cytometry data was analyzed using FlowJo software. Briefly, the barcode population was gated in the FSC vs. SSC plot and further gated in the 2D-plot with 530/30 nm and 585/42 nm BP filters (corresponding to the barcode signal). The median intensity of barcodes in the histogram of the 661/16 nm BP filter (corresponding to the Alexa Fluor 647 reporter probe signal) was then recorded and normalized to the highest signal from the 4° C. solution-based or tablet-based sandwich assay. The median and the median absolute deviation was then plotted in GraphPad Prism software such that the amount of target DNA used in the sandwich assay was the independent variable and normalized fluorescence intensity from the reporter probe was the dependent variable. A regression analysis (which included whether the line is significantly different from zero) of the curves were then analyzed at each time point using GraphPad Prism. For the synthetic RPA thermal stability studies, a Mann-Whitney test was conducted for each time point using SPSS software. Clinical studies (benchtop). Similar to the thermal stability studies, flow cytometry data was analyzed using FlowJo software. We normalized the median fluorescence intensity from each of the barcodes to the highest median signal. We analyzed the pooled healthy patient samples and the HBV samples using a Mann-Whitney H test using SPSS software. Clinical studies (point-of-care). We uploaded the smartphone images of barcodes onto a desktop computer and analysed using MATLAB. For each image, we used a Hough transform to determine barcode locations. The difference between the average intensity over the extracted location and surrounding background was then attributed each of the respective barcodes. Pooled median background subtracted fluorescent intensity for each sample replicate (2 replicates each) of healthy and we used the HBV-infected data for statistical analysis using SPSS software. Similar to data analysis performed for flow cytometry, the healthy patient samples and we analyzed HBV samples using a Mann-Whitney H test on SPSS.

### 2. Results

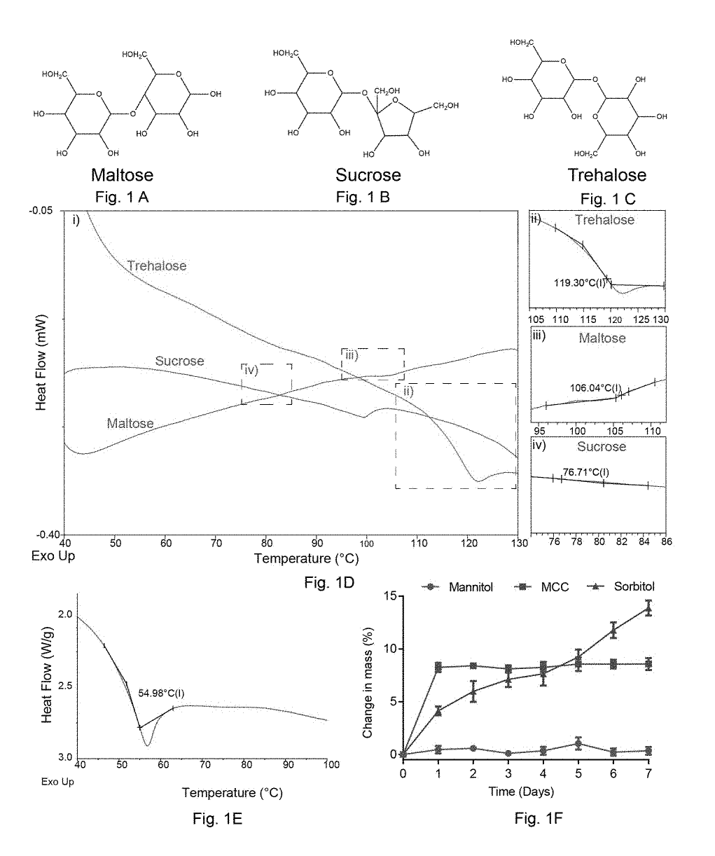

Principle of chemical interactions between excipients (or additives) and diagnostic reagents. Diagnostic reagents in solution can be subjected to hydrolysis, oxidation and other enzymatic degradations due to their high molecular mobility in water. The molecular mobility of reagents can be minimized by removing the surrounding water content, via lyophilization. Although lyophilization has the potential to stabilize labile reagents, the process can generate excessive stresses that can further degrade diagnostic reagents. Sugars can be added to stabilize the reagents during lyophilization. Although, the selection of the type of sugar molecule is critical to this stabilization process. For optimal stabilization, we wanted to select a sugar with a high glass transition temperature (T_{g}). The plasticizing effects of water can lower the T_{g}s of sugars during shipping and storage of reagents at high humidity, leading to increased molecular mobility and consequently reagent degradation. Thus, we analyzed the T_{g}s of commonly used sugars, maltose, sucrose and trehalose, using differential scanning calorimetry (DSC) to determine the optimal stabilizer (FIGS. 1A to 1C). We selected trehalose to stabilize reagents during lyophilization, owing to its high Tg (FIG. 1D i-iv). Even after moisture uptake, the glass transition temperature of trehalose remained above the storage temperature of reagents, allowing reagents to have minimal mobility (FIG. 1E). In addition, among the disaccharides, trehalose has the fewest intramolecular hydrogen bonds. Intramolecular hydrogen bonds restrict the movements of the ring structures in polysaccharides. The rigidity of polysaccharides can limit hydrogen bonding between the remaining hydroxyl groups and polar groups of biomolecules. The molecular flexibility of trehalose on the other hand, allows it to replace the hydration shell around biomolecules, preserving the three-dimensional conformations of reagents. Lastly, trehalose is a kosmotrope that disrupts the tetrahedral hydrogen-bonded network of water. Trehalose has a larger hydration number (the average number of water hydrogen bonded to the disaccharide), which allows water to be ordered around trehalose instead of the biomolecules. The “destructuring” of water molecules around biomolecules minimizes ice crystallization during lyophilization, thereby protecting the biomolecules from damage¹¹.

Next, in order to directly compress the reagents into easy-to-handle tablets, the reagents needed to be mixed with highly compactible powders. Thus, we screened spray-dried mannitol, D-sorbitol and microcrystalline cellulose (MCC) to act as the bulking agents for the reagent tablets. To maintain the T_{g} of trehalose and to provide stability to reagents, the bulking agent needed to be inert and provide a moisture-free environment. We stored tablets developed from spray-dried mannitol, D-sorbitol and MCC for one week at 80% relative humidity (RH) and examined the changes in mass due to moisture uptake (FIG. 1F). Among the three excipients, sorbitol had the greatest change in mass due to moisture uptake. Although sorbitol is a diastereomer of mannitol, it is highly hygroscopic and absorbs moisture from the environment. The significant difference in hydration between the two isomers is attributed to the intermolecular forces in the solid-state. Unlike mannitol, sorbitol is able to form stable co-crystals with water at ambient conditions. The stability of the sorbitol-water co-crystals is due to a combination of both favorable molecular conformations as well as intermolecular cohesion relative to anhydrous sorbitol. Although MCC also exhibited low moisture absorption relative to sorbitol, reactive impurities found in MCC, such as trace levels of glucose, can react with amino groups of proteins in a Mail-lard reaction. In a typical reaction, the glycosidic hydroxyl groups of glucose is replaced by the amine, producing glycosamine and other by-products. Therefore, we selected spray-dried mannitol as the bulking agent of reagent tablets, owing to its inertness and non-hygroscopicity (FIG. 1F).

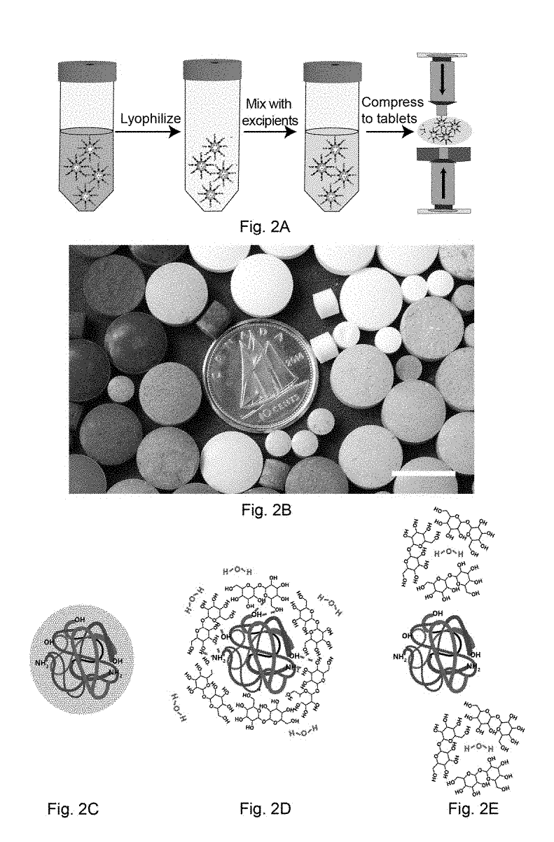

Development of reagent tablets. Tablets were developed by first lyophilizing the diagnostic reagents, followed by mixing with additives and lastly compressing the mixture into individual tablets. We first removed the surrounding water content by lyophilizing the reagents with 15% trehalose in 0.05% Tween 20. Trehalose, a non-hygroscopic cryoprotectant, provides a highly viscous glassy matrix to limit molecular mobility and stabilizes the reagents through hydrogen bonding. After lyophilization, we mixed the reagents with three excipients: spray-dried mannitol, croscarmellose sodium and sodium stearyl fumarate. We used spray-dried mannitol, a non-hygroscopic sugar-alcohol, as the bulking agent of the tablet. Its non-hygroscopic nature can limit the interaction of the reagents with surrounding water vapor and further prevent degradation. We also included croscarmellose sodium to help disintegrate the tablet and sodium stearyl fumarate to help lubricate the rotary press die walls for ease of tablet ejection (FIG. 2A). After mixing the reagents and excipients, we compressed the mixture with a rotary tablet press using a compression force of 800 N. We can generate customizable and readily dissolvable tablets (<30 seconds) at a rate of more than 300 tablets per minute (FIG. 2B). In addition, the tablets can stabilize reagents by using the vitrification properties of trehalose as well as by providing a moisture-free barrier environment (FIGS. 2C to 2E).

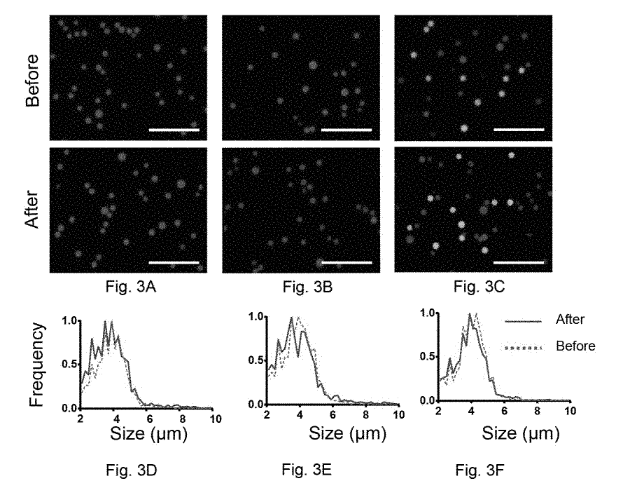

Characterizing reagents in tablets using a model system: quantum dot (QD) barcodes. To characterize the properties of diagnostic reagents in tablets, we used a multi-step diagnostic assay. This assay uses QD barcodes, polymer microspheres encapsulated with QDs, that can detect a multitude of molecules in biological fluids.¹ QD barcodes can be characterized with multiple techniques, including fluorescence microscopy and flow cytometry, allowing for the ease of characterizing reagents in tablets. In a typical assay, multiple reagents, including the buffer, fluorescent detection probes and target DNA are all mixed with DNA-conjugated QD barcodes. The DNA-conjugated polymer microspheres, target DNA and the fluorescent detection probe form a sandwich complex. The signal from the fluorescent DNA probes and QD barcodes can then be detected to identify the presence and type of the target, respectively.² We developed QD barcode tablets to first characterize the physical properties of diagnostic reagents post-tableting. Overall when barcodes were encapsulated in compressed tablets, their structure, size and fluorescence intensity were maintained (FIGS. 3A to 3F). The process of tableting therefore does not change the physical properties of the reagents.

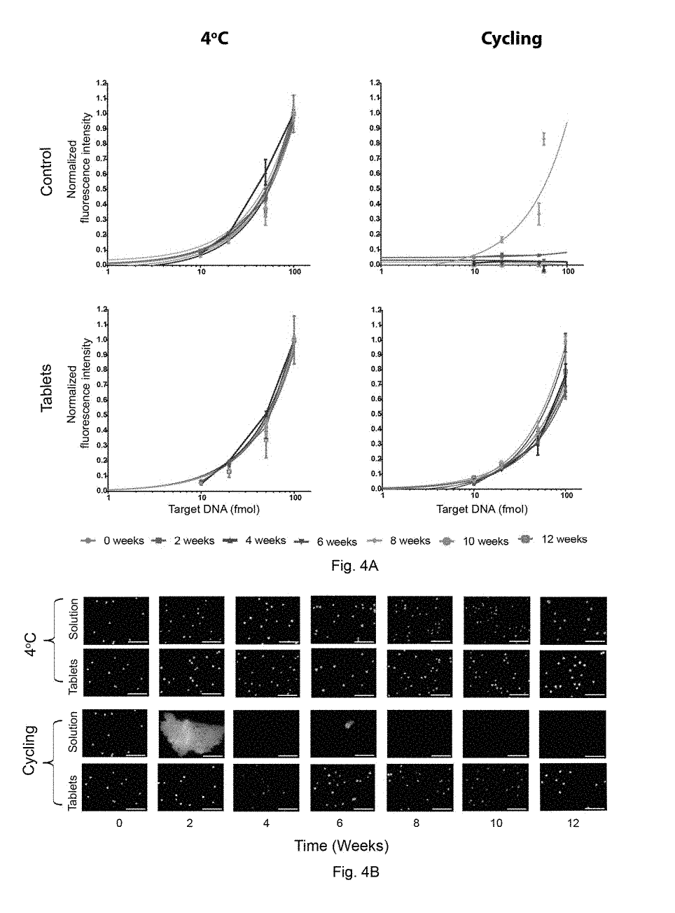

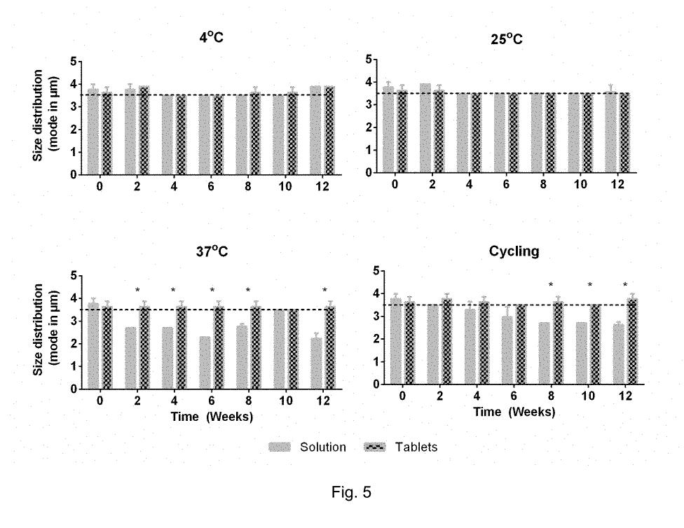

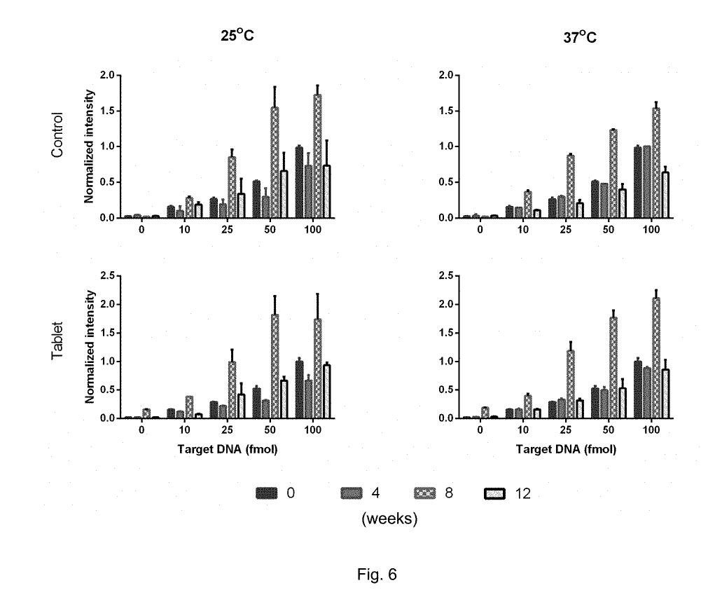

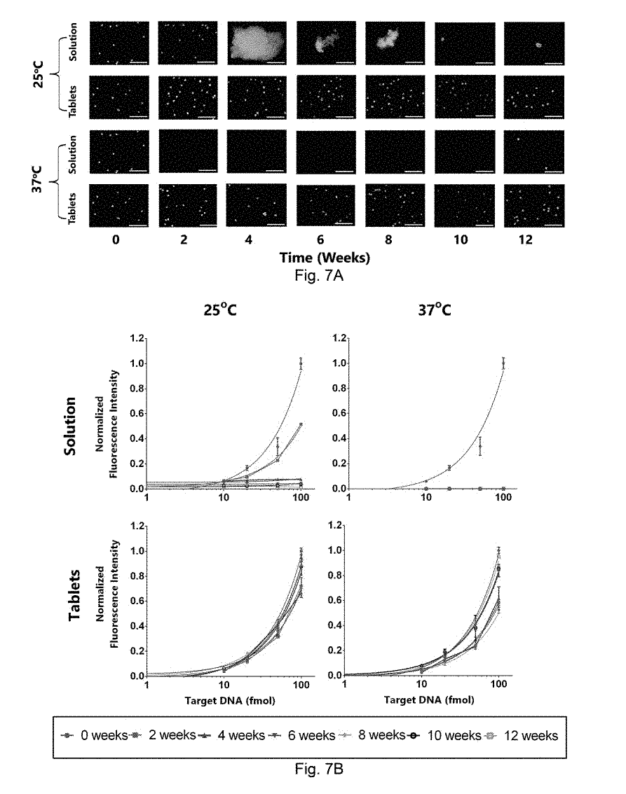

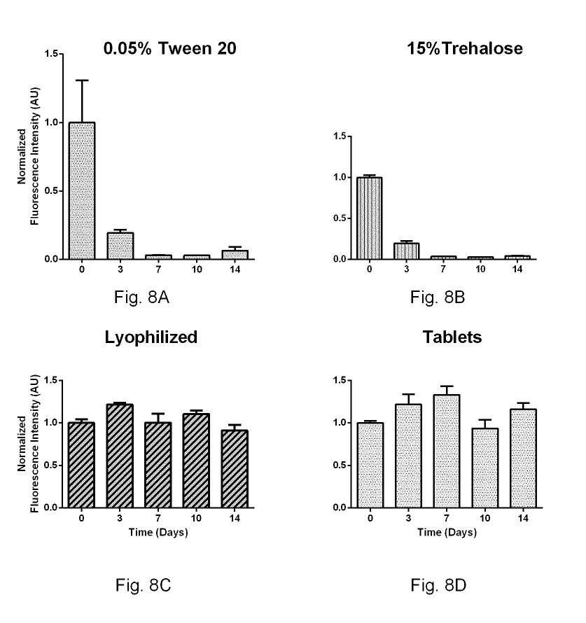

Characterizing the thermal stability of reagent tablets using QD barcodes. Next, we evaluated the thermal stability of the barcode assay components. We compared the fluorescence, analytical performance and structure of barcodes after storage in solution or tablets at various temperatures for 12 weeks (FIGS. 4, 5 and 6, Tables 1-2). QD barcodes in tablets and in solution were stored at 25° C. and 37° C. to mimic storing diagnostic reagents at room temperature and tropical climates. QD barcodes stored in solution aggregated within 4 weeks at 25° C. and completely degraded within 2 weeks at 37° C. (FIG. 7A). These properties impact the performance of the barcodes, as aggregation affects the identification of the pathogens and the analytical performance. In contrast, when QD barcodes were stored in tablets, the fluorescence and the structure of the microspheres were maintained for up to 12 weeks at 25° C. and 37° C. (FIG. 7A). In order to evaluate the diagnostic capabilities of the QD barcodes tablets, we also conducted a single-plex sandwich assay to monitor the effect of the barcode's storage temperature on the analytical sensitivity of the assay (FIG. 7B), or the assay's ability to detect a change in concentration. At each time-point, we added the barcode tablet (with 10-30 second dissolution) to a fluorescent DNA detection probe and solutions of known concentrations of target DNA (0 to 200 fmol) for 30 minutes, and then used a magnetic field to purify the barcodes. We show that barcodes were able to consistently detect and differentiate between different concentrations of the target sequence when they were stored in tablets (FIG. 7B) at either 25° C. and 37° C. (p=0.0008 and 0.0006, respectively). In contrast, by 4 weeks at 25° C. and by 2 weeks at 37° C., the non-tableted barcodes in solution could not differentiate between different concentrations of the target DNA (FIG. 7B 4 weeks: p=0.206). The polystyrene co-polymer of the barcodes is held together by non-covalent forces that can be easily disrupted by temperature, resulting in surface morphological changes, agglomeration and eventually complete degradation of the microspheres¹². Tablets can circumvent this by encapsulating lyophilized reagents and by minimizing contact with solvents such as water that may accelerate this degradation process. Lyophilization removes water from the microspheres' surrounding environment; limiting molecular mobility and consequently, QD barcode degradation (FIGS. 8A to 8D). Encapsulation in trehalose limits the interaction of the reagents with residual water and protects the microspheres from ice formation during the freezing stage. According to the water replacement theory, trehalose hydrogen bonds with the microbeads and acts as a substitute for water; thus maintaining their structure. Other theories postulate that trehalose forms a glassy matrix to limit molecular mobility and degradation. The high glass transition temperature (T_{g}) of trehalose also allows the lyophilized samples to be stored at elevated temperatures. Finally, our compressed method can increase the densification of the powder blends, which can potentially reduce contact of the reagents with water vapour—protecting the quantum dot fluorescence from the ambient environment.

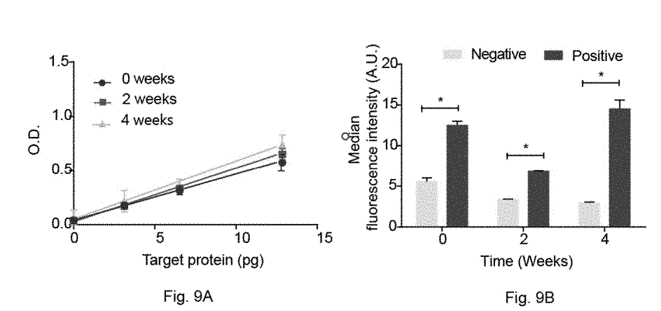

Application of the tablets with other diagnostic systems. To show that our tableting approach can be applied to other diagnostic assays, we evaluated tableting reagents of protein-based and genetic tests. First, we chose an enzyme-based immunoassay detecting CXCL5, a chemokine implicated in prostate cancer¹³, as a model protein-based diagnostic test. CXCL5 is detected when a color change occurs as a result of avidin-horse radish peroxidase (avidin-HRP) binding to an immobilized antibody-CXCL5-biotinylated antibody complex. To demonstrate the applicability of using tablets for antibodies and enzymes, we encapsulated avidin-HRP in compressed tablets. We then examined the stability of avidin-HRP in tablets for a period of 4 weeks at 25° C. At each time point, we created a standard curve by adding different concentrations of CXCL5 to a plate of immobilized and biotinylated antibodies. Avidin-HRP tablets were then added to create a color change. At each time point, avidin-HRP remained stable and functional in compressed tablets for up to 4 weeks (FIG. 9A, Time 0: p<0.0001, Time 2: p =0.0005, and Time 4: p=0.0023).

Next, we chose a nucleic acid amplification technique, called recombinase polymerase amplification (RPA), as a model genetic test. This technique uses recombinase proteins, which form a nucleoprotein complex with primers, to facilitate strand exchange at homologous sequences of the template DNA. As single-stranded binding proteins (SSB) stabilize this complex, a DNA polymerase extends the template of interest to provide exponential amplification¹⁴. To demonstrate the applicability of using tablets for DNA and proteins, we encapsulated primers (Table 3) and all protein components required for RPA including recombinase, polymerase, single-stranded binding proteins and other co-factors, in a compressed tablet. We then stored the tablets at 25° C. for 4 weeks. At each time point, the stability of RPA components in tablets was evaluated by amplifying 10⁷ synthetic copies of DNA (Table 3). Overall, RPA proteins and primers remained stable at room temperature for up to 4 weeks in tablets (FIG. 9B, 4 weeks: p=0.05). We can therefore easily adapt this tabletting technology for other diagnostic platforms without adversely affecting biomolecular reagents over time.

To further simplify the amplification process and minimize user intervention, primers for RPA were modified to eliminate the denaturation step (which is required to produce single-stranded DNA for hybridization in the QD barcode assay). Primers were modified with a 3′ C3 spacer and an additional hybridization sequence. With these modified primers, DNA extension ceases when polymerase encounters the C3 spacer, leaving the hybridization sequence as single-stranded overhangs at either ends of the amplicon. These overhangs, then hybridize with the nucleic acid conjugated QD barcodes and fluorescent reporter probes to identify the presence and type of pathogen in the sample.

Clinical testing of HBV with multiple tablets. Next, we evaluated whether our tableting technology can be used to simplify a multi-step assay for screening patient samples. We developed three tablets: an RPA tablet, a QD barcode tablet and a tablet containing a fluorescent reporter probe (DNA probe with Alexa Fluor 647) for clinical HBV samples (Tables 1 & 4). We collected samples from HBV+ patients and used a combination of the genetic test and the barcode technology to demonstrate genetic detection of healthy and HBV+ patients. We first extracted nucleic acids from serum samples. We then added the RPA tablet for amplification, then denatured the sample at 100° C., and added the QD barcode tablet (containing barcodes for the HBV target) as well as the reporter probe tablet for the HBV target (FIG. 10A). We screened three healthy and three HBV patient samples using all three reagent tablets and analyzed the results with a bench-top flow cytometer and a point-of-care smartphone device (FIG. 10A, item 4). With the use of reagent tablets, we were able to differentiate between healthy and HBV patient samples using bench-top and point-of-care instrumentation (FIG. 10B-C, p<0.0001). We were therefore able to demonstrate that our tableting strategy can (a) simplify multi-step diagnostic assays to (b) screen for patient samples.

### CONCLUSIONS

We stabilized and simplified multi-step diagnostic assays by using a high-throughput compression strategy to develop reagent tablets. Reagent tablets offer an inexpensive (Table 5) means for providing thermal stability to reagents. This technology allows areas with high burden of diseases but low infrastructural capabilities to also have access to medical diagnostics. Reagents can degrade during shipping and storage in tropical environments. This can compromise test results and ultimately lead to misdiagnoses and poor patient outcomes. The tableting system of the present invention is able to stabilize diagnostic reagents at elevated temperatures. To develop tablets, first reagents were lyophilized with trehalose. Trehalose acted as a cryoprotectant, shielding the constituents from ice crystals. Due to the protective properties of trehalose, a variety of reagents, ranging from antibodies to polymer micro-spheres, were able to be lyophilized without degradation. Following lyophilization, diagnostic reagents were encapsulated in tablets without altering the structural and functional properties of the reagents. We were able to store polymer microspheres at elevated temperatures for 3 to 6 times longer than when stored in solution. We were also able to extend this technology to other types of assays which have antibodies, enzymes, and DNA, without adversely affecting the biomolecules. Furthermore, once dissolved, the tablets did not interfere with the diagnostic assays conducted. It is therefore possible to easily apply this technology and formulation to a myriad of other molecular and chemical diagnostic assays, such as protein and nucleic acid tests. In addition to providing thermal stability, reagent tablets are also easy-to-handle and provide pre-measured quantities of reagents without needing additional packaging consumables. As a result, we were able to reduce user-intervention when conducting multi-step assays. We screened healthy and HBV+ clinical samples by adding a series of tablets and used both bench-top and point-of-care smartphone technology for detection. Reagent tablets can therefore be used to screen clinical specimens in advanced as well as point-of-care facilities.

Compressed tablets offer a variety of advantages for pharmaceutical drug encapsulation, including: (a) providing stability, (b) cost-effectiveness, and (c) fast dissolution times. The present invention exploits these advantages and apply this platform to address barriers limiting the translation of medical diagnostic assays. Although it is possible to simply lyophilize the molecular reagents, their subsequent encapsulation in tablets provides an easy and simple way of developing pre-packaged reagents. In the absence of pre-packaged tablets, lyophilized reagents need to be resuspended, aliquoted, stored and then measured before use, increasing the risk of introducing human error and inter-operator variability. Others have demonstrated packaging lyophilized reagents in consumables¹⁵, such as Eppendorf tubes. However, this increases the cost of the assay and limits their translation to resource-poor settings (˜$20 for 100 tubes vs. $0.16 for 100 tablets). Although there is demonstration of development of reagent tablets, these were developed by exploiting the film-forming properties of pullulan, rather than through direct compaction of reagents^{16,17}. It is not clearly evident whether these reagent tablets can be kept at elevated temperatures (>25° C.) for prolonged periods of time.

In order to expedite the clinical translation of these technologies, it is important to demonstrate their performance in complex biological matrices. We have demonstrated the suitability of translating our tableting technology to resource-limited settings by showing the ease of using tablets, in combination with a smartphone instrumentation camera, for screening clinical samples. Prior to using tableted reagents, the workflow for a diagnostic assay required a number of experts ranging from phlebotomists and technicians to clinicians. The lack of availability of such experts, especially in remote locations, has hindered the progression of diagnostics in resource-poor areas. State-of-the-art diagnostics therefore have not made the jump from the bench to the clinic. By introducing reagent tablets, we have simplified the workflow such that a lay person can still perform complex diagnostics with everyday household items, such as with a cup of water to dissolve the tablets or a stove to heat reactions.

While we demonstrate the utility of tableting reagents for medical diagnostic applications, we can extend this technology for detecting contaminants in the environment (lakes and rivers), or food-borne pathogens in produce (meats, dairy). Mixing a reagent tablet with a sample of contaminated water or a tissue sample to identify contaminants and pathogens. The present invention enables the accessibility of assays by simplifying complex reagent-based sensors. An end-user can add a sequence of color-coded tablets to the sample of interest, then to wait for a period of time and finally to visualize the result by eye by a handheld smartphone camera. The present invention can bridge the gap between the development of diagnostic tests and their subsequent translation to re-source-poor areas.

### TABLES

TABLE 2
Capture probes, target sequences and
reporter probe used for synthetic
single-plex and multi-plex sandwich assay

| Infection | Capture probe | Target DNA |
|---|---|---|
| type | sequence (5′-3′) | sequence (5′-3′) |
| HIV | NH₂C6 GAGACCATCA | CGGCGATGAATACCTA |
| | ATGAGGAAGCTGCAGA | GCACACTTACTAATCC |
| | ATGGGAT | CATTCTGCAGCTTCCT |
| | (SEQ ID NO: 1) | CATTGATGGTCTC |
| | | (SEQ ID NO: 2) |
| HBV | NH₂C6 TCAGAAGGCA | CGGCGATGAATACCTA |
| | AAAAAGAGAGTAACT | GCACACTTACTAAGTT |
| | (SEQ ID NO: 3) | ACTCTCTTTTTTGCCT |
| | | TCTGA |
| | | (SEQ ID NO: 4) |
| Syphilis | NH₂ C6 GACAATGCT | CGGCGATGAATACCTA |
| | CACTGAGGATAGT | GCACACTTACTAACTA |
| | (SEQ ID NO: 5) | TCCTCAGTGAGCATTG |
| | | TC |
| | | (SEQ ID NO: 6) |
| Gonorrhea | NH₂C6 CCAATATCGG | N/A |
| | CGGCC | |
| | (SEQ ID NO: 7) | |

*Universal reporter probe: /5Alex647N/TAA GTG TGC TAG GTA TTC ATC GCC G3 (SEQ ID NO: 8)--’

TABLE 3
RPA primers, capture probes,
target and reporter probe used for
validation of compressed tablets

| DNA ID | Sequence (5′-3′) |
|---|---|
| HBV forward | TGTT GACAAGAATC CTCACAATAC |
| primer | CACAGAGTC (SEQ ID NO: 9) |
| HBV reverse | CGAA TTTTGGCCAG GACACACGGG TGTTCC |
| primer | (SEQ ID NO: 10) |
| Capture probe | NH₂C6 TTTTTTTTCGAATTTTG |
| sequence | GCCAGGACACACGGGTGTTCCCCCTAGAAAAT |
| | TGAGA (SEQ ID NO: 11) |
| Target | TGTTGACAAGAATCCTCACAATACCACAGAGT |
| amplicon | CTAGACTCGTGGTGGACTTCTCTCAATTTTCTA |
| | GGGGGAACACCCGTGTGTCCTGGCCAAAATTC |
| | G (SEQ ID NO: 12) |
| Reporter | AGTCT AGACTCTGTG GTATTGTGAG |
| probe | GATTCTTGTC AACA A647 |
| | (SEQ ID NO: 13) |

TABLE 4
Viral loads of HBV clinical samples

| Sample | | |
|---|---|---|
| ID | IU/mL | Copies/μL |
| 6 | 1.69E+08 | 9.84E+05 |
| 7 | 1.70E+08 | 9.89E+05 |
| 8 | 1.70E+08 | 9.89E+05 |

TABLE 5
Cost analysis (for 10 million QD barcodes) of the reagents necessary for
manufacturing one tablet
Reagents

| | | | | Required | Cost |
|---|---|---|---|---|---|
| | | | | (in grams) | per |
| | | | | for one 50 | 50 mg |
| Item | Supplier | Catalogue # | Unit Price | mg tablet | tablet |
| D-(+)- | Bioshop | TRE222.25 | $44.96/25 g | 2.70E−04 | $0.0005 |
| trehalose | | | | | |
| dihydrate | | | | | |
| Mannogem | SPI | | $16.82/400 kg | 4.63E−02 | $0.0000 |
| EZ | Pharma | | | | |
| (Spray- | | | | | |
| dried | | | | | |
| mannitol) | | | | | |
| Croscar- | Tablet | cros50 | $6.80/50 g | 3.00E−03 | $0.0004 |
| mellose | Press | | | | |
| sodium | Club | | | | |
| Sodium | Tablet | ssfl | $21.95/20 g | 5.00E−04 | $0.0005 |
| stearyl | Press | | | | |
| fumarate | Club | | | | |
| Tween 20 | BioShop | TWN510.500 | $10.45/500 mL | 9.90E−03 | $0.0002 |
| Total | | | | | $0.0016 |

TABLE 6

| | 0.05% Tween | 15% Trehalose | Lyophilized | Tablets |
|---|---|---|---|---|
| 0.05% Tween | N/A | 1.00 × 10¹ | 8.40 × 10⁻⁹ | 3.82 × 10⁻⁹ |
| | | N.S. | **** | **** |
| 15% Trehalose | 1.00 × 10¹ | N/A | 8.23 × 10⁻⁹ | 3.75 × 10⁻⁹ |
| | N. S. | | **** | **** |
| Lyophilized | 8.40 × 10⁻⁹ | 8.23 × 10⁻⁹ | N/A | 7.7 × 10⁻² |
| | **** | **** | | N. S. |
| Tablets | 3.82 × 10⁻⁹ | 3.75 × 10⁻⁹ | 7.7 × 10⁻² | N/A |
| | **** | **** | N. S. | |

### NON-PATENT REFERENCES

(1) Chen, K.; Chou, L. Y. T.; Song, F.; Chan, W. C. W. Nano Today 2013, 8 (3), 228.
(2) Giri, S.; Sykes, E. a; Jennings, T. L.; Chan, W. C. W. ACS nano 2011, 5 (3), 1580.
(3) Fournier-Bidoz, S.; Jennings, T. L.; Klostranec, J. M.; Fung, W.; Rhee, A.; Li, D.; Chan, W. C. W. Angewandte Chemie (International ed. in English) 2008, 47 (30), 5577.
(4) Ming, K.; Kim, J.; Biondi, M. J.; Syed, A.; Chen, K.; Lam, A.; Ostrowski, M.; Rebbapragada, A.; Feld, J. J.; Chan, W. C. W. ACS Nano 2015, 9 (3), 3060.
(5) Haque, K.; Kawai, K.; Suzuki, T. Carbohydrate Research 2006, 341, 1884.
(6) Willart, J. F.; Gusseme, A. De; Hemon, S.; Descamps, M.; Leveiller, F.; Rameau, A. J. Phys. Chem. B 2002, 106, 3365.
(7) Dabbousi, B. O.; Mikulec, F. V; Heine, J. R.; Mattoussi, H.; Ober, R.; Jensen, K. F.; Bawendi, M. G. J. Phys. Chem. B 1997, 101 (46), 9463.
(8) Bae, W. K.; Nam, M. K.; Char, K.; Lee, S. Chem. Mater. 2008, 20, 5307.
(9) Hou, Y.; Xu, Z.; Sun, S. Angewandte Chemie (International ed. in English) 2007, 46, 6329.
(10) Kim, J.; Biondi, M. J.; Feld, J. J.; Chan, W. C. W. ACS Nano 2016, 10 (4), 4742.
(11) Lerbret, A.; Bordat, P.; Affouard, F.; Descamps, M.; Migliardo, F. J. Phys. Chem. B 2005, 109 (21), 11046.
(12) Zolnik, B. S.; Leary, P. E.; Burgess, D. J. Journal of Controlled Release 2006, 112, 293.
(13) Begley, L. A.; Kasina, S.; Mehra, R.; Adsule, S.; Admon, A. J.; Lonigro, R. J.; Chinnaiyan, A. M.; Macoska, J. A. Neoplasia 2008, 10 (3), 244.
(14) Piepenburg, O.; Williams, C. H.; Stemple, D. L.; Armes, N. a. PLoS biology 2006, 4 (7), e204.
(15) Pardee, K.; Slomovic, S.; Nguyen, P. Q.; Lee, J. W.; Donghia, N.; Burrill, D.; Ferrante, T.; McSorley, F. R.; Furuta, Y.; Vernet, A.; Lewandowski, M.; Boddy, C. N.; Joshi, N. S.; Collins, J. J. Cell 2016, 167 (1), 248.
(16) Jahanshahi-anbuhi, S.; Pennings, K.; Leung, V.; Liu, M.; Carrasquilla, C.; Kannan, B.; Li, Y.; Pelton, R.; Brennan, J. D.; Filipe, C. D. M. Angewandte Chemie (International ed.) 2014, 53, 6155.
(17) Jahanshahi-anbuhi, S.; Kannan, B.; Leung, V.; Pennings, K.; Liu, M.; Carrasquilla, C.; White, D.; Li, Y.; Pelton, R. H.; Brennan, J. D.; Filipe, C. D. M. Chemical Science 2016, 7, 2342.

Through the embodiments that are illustrated and described, the currently contemplated best mode of making and using the invention is described. Without further elaboration, it is believed that one of ordinary skill in the art can, based on the description presented herein, utilize the present invention to the full extent.

Although the description above contains many specificities, these should not be construed as limiting the scope of the invention, but as merely providing illustrations of some of the presently embodiments of this invention. The following claims are provided to add additional clarity to this disclosure. Future applications claiming priority to this application may or may not include the following claims, and may include claims broader, narrower, or entirely different from the following claims.

What is claimed is:

1. A solid diagnostic product comprising reagents necessary for the detection of a target of interest.

2. The solid diagnostic product of claim 1, wherein the reagents are of quantum dot barcodes conjugated to a capture probe having affinity for the target of interest.

3. The solid diagnostic product of claim 1, wherein the reagents are lyophilized in a sugar having high Tg and mixed with spray-dried mannitol and at least one acceptable carrier or excipient.

4. The solid diagnostic product of claim 1, wherein the solid diagnostic product is color-coded to mediate a diagnostic sequence.

5. The solid diagnostic product of claim 1, wherein the target of interest is a nucleic acid of interest, and wherein the solid diagnostic product includes:
(a) a layer or tablet having reagents for amplifying a nucleic acid sequence of interest;
(b) a layer or tablet having QD barcodes conjugated to a capture probe having affinity for the nucleic acid sequence of interest;
(c) a layer or tablet having a reporter probe for the nucleic acid sequence of interest, and optionally
(d) a layer or tablet having a reporter probe for controls.

6. The solid diagnostic product of claim 5, wherein the (a) layer or tablet comprises reagents for a recombinase polymerase amplification (RPA).

7. The solid diagnostic product of claim 6, wherein the reagents in the (a) layer or tablet are for RPA are: RPA recombinase, polymerase, single-stranded binding proteins (SSBs), and other co-factors and primers for the nucleic acid sequence of interest.

8. The solid diagnostic product of claim 7, wherein the primers reagents in the (a) layer or tablet include a 3′ C3 spacer between a hybridization and a primer sequence.

9. The solid diagnostic product of claim 5, wherein the layers or tablets are color-coded to mediate a diagnostic sequence.

10. The solid diagnostic product of claim 1, wherein the solid diagnostic product includes the reagents lyophilized with trehalose and Iron-EDTA, mixed with spray-dried mannitol and sodium stearyl fumarate.

11. The solid diagnostic product of claim 1, wherein the solid diagnostic product includes:
(a) a layer or tablet having a detectable primary ligand to the target of interest; and
(b) a layer or tablet having a detectable secondary ligand to a complex formed by the primary ligand and the target of interest.

12. The solid diagnostic product of claim 11, wherein the detectable primary ligand is a primary antibody-conjugated quantum dot (QD) barcode.

13. The solid diagnostic product of claim 11, wherein the detectable secondary ligand is an AlexaFluor-647 dye conjugated secondary antibody.

14. The solid diagnostic product of claim 1, wherein the solid diagnostic product is a multi-layered tablet comprising:
(a) a top, fast-release layer having a detectable primary ligands to the target of interest,
(b) a core layer having secondary detection ligands to the complex formed between the primary ligand and the target of interest, and
(c) a barrier layer between the top and the core layers configured to delay the core layer from releasing the secondary detection ligands.

15. The solid diagnostic product of claim 1, wherein the solid diagnostic product is a multi-layered tablet comprising:
(a) a top, fast release layer having reagents for a recombinase polymerase amplification (RPA);
(b) a core layer having QD barcodes and reporter probes; and
(c) a barrier layer between the top and the core layers configured to delay the core layer from releasing the QD barcode components.

16. A kit for diagnosis of a condition produced by a pathogen, the kit comprising:
(a) a first solid diagnostic product comprising reagents for amplifying a DNA sequence characteristic of the pathogen,
(b) a second solid diagnostic product comprising quantum dot barcodes conjugated to a capture probe having affinity for the same DNA sequence of the pathogen, and
(c) a third solid diagnostic product comprising a labelled reporter probe having affinity for the same DNA sequence of the pathogen.

17. The kit of claim 16, wherein the kit further comprises another solid diagnostic product comprising a reporter probe for controls.

18. The kit of claim 16, wherein the reagents for amplifying the DNA sequence are reagents for a recombinase polymerase amplification.

19. The kit of claim 16, wherein said solid diagnostic product is provided in the form of a multi-layered tablet wherein each layer represents each of the solid diagnostic products.

20. A method of detecting a biological target of interest in a biological sample, the method comprising contacting the biological sample with the solid diagnostic product of claim 1 and analyzing the biological sample for a presence of the target in the biological sample.


SEQUENCE LISTING (13 sequences)

SEQ ID NO: 1 - DNA, 33 bp, Artificial Sequence
  Synthetic
  modified_base (1)..(1): NH2C6
gagaccatcaatgaggaagctgcagaatgggat

SEQ ID NO: 2 - DNA, 61 bp, Artificial Sequence
  Synthetic
cggcgatgaatacctagcacacttactaatcccattctgcagcttcctcattgatggtctc

SEQ ID NO: 3 - DNA, 25 bp, Artificial Sequence
  Synthetic
  modified_base (1)..(1): NH2C6
tcagaaggcaaaaaagagagtaact

SEQ ID NO: 4 - DNA, 53 bp, Artificial Sequence
  Synthetic
cggcgatgaatacctagcacacttactaagttactctcttttttgccttctga

SEQ ID NO: 5 - DNA, 22 bp, Artificial Sequence
  Synthetic
  modified_base (1)..(1): NH2C6
gacaatgctcactgaggatagt

SEQ ID NO: 6 - DNA, 50 bp, Artificial Sequence
  Synthetic
cggcgatgaatacctagcacacttactaactatcctcagtgagcattgtc

SEQ ID NO: 7 - DNA, 15 bp, Artificial Sequence
  Synthetic
  modified_base (1)..(1): NH2C6
ccaatatcggcggcc

SEQ ID NO: 8 - DNA, 25 bp, Artificial Sequence
  Synthetic
  modified_base (1)..(1): 5Alex647N
taagtgtgctaggtattcatcgccg

SEQ ID NO: 9 - DNA, 33 bp, Artificial Sequence
  Synthetic
tgttgacaagaatcctcacaataccacagagtc

SEQ ID NO: 10 - DNA, 30 bp, Artificial Sequence
  Synthetic
cgaattttggccaggacacacgggtgttcc

SEQ ID NO: 11 - DNA, 54 bp, Artificial Sequence
  Synthetic
  modified_base (1)..(1): NH2C6
ttttttttcgaattttggccaggacacacgggtgttccccctagaaaattgaga

SEQ ID NO: 12 - DNA, 98 bp, Artificial Sequence
  Synthetic
tgttgacaagaatcctcacaataccacagagtctagactcgtggtggacttctctcaattttctagggggaacacccgtgtgtcctggccaaaattcg

SEQ ID NO: 13 - DNA, 39 bp, Artificial Sequence
  Synthetic
  modified_base (39)..(39): A647
agtctagactctgtggtattgtgaggattcttgtcaaca